(19) United States
(12) Patent Application Publication
(10) Pub. No.: US 2009/0148920 A1
(43) Pub. Date: Jun. 11, 2009

(54) Integrated glyceride extraction and biodiesel production processes

(75) Inventor: David James Schreck, Lake City, MN (US)

Correspondence Address: PAULEY PETERSEN & ERICKSON, 2800 WEST HIGGINS ROAD, SUITE 365, HOFFMAN ESTATES, IL, 60169, US

(73) Assignee: Best Energies, Inc.

(21) Appl. No.: 12/315,518

(22) Filed: Dec. 4, 2008

Related U.S. Application Data
(60) Provisional application No. 61/005,518, filed on Dec. 5, 2007.
(60) Provisional application No. 61/007,309, filed on Dec. 12, 2007.
(60) Provisional application No. 61/062,959, filed on Jan. 29, 2008.

(51) Int. Cl. C12P 7/62; C07C 67/48

(52) U.S. Cl. 435/135; 560/191

(57) ABSTRACT

Biodiesel is used to extract glycerides from biomass derived sources and the extractant containing biodiesel and glycerides is subjected to ester-forming conditions including the presence of lower alkanol to produce biodiesel, a portion of which is used for the extraction of glycerides.

### CROSS REFERENCE TO RELATED APPLICATION(S)

This application claims the benefit of each of U.S. Provisional Application No. 61/005,518, filed 5 Dec. 2007; U.S. Provisional Application No. 61/007,309, filed 12 Dec. 2007; and U.S. Provisional Application No. 61/062,959, filed 29 Jan. 2008.

### FIELD OF THE INVENTION

This invention pertains to integrated processes for the recovery of glycerides from feedstocks, and particularly to such processes wherein the recovered glycerides are suitable for use as a feed for making biodiesel.

### BACKGROUND TO THE INVENTION

Biodiesel comprises lower alkyl esters, usually methyl ester, of fatty acids of 16 to 20 carbon atoms. The feedstock for making biodiesel are oils from biomass containing triglycerides such as rape seed, soybean, cotton seed, safflower seed, castor bean, olive, coconut, palm, corn, canola, jatropha, rice bran, tobacco seed, and animal sources. The feedstock is subjected to esterification conditions, usually either acid or base catalyzed, in the presence of lower alkanol to form the alkyl ester of fatty acid and produce as a co-product, glycerin.

Numerous techniques have been proposed to recover glycerides from biomass, and the particular technique most attractive may vary depending upon the feedstock. For grains such as soybean and corn, extraction of oils using hexane is practiced. Although oils can be removed without the use of solvent, the recovery of oils is reduced and the oil content of the residue, e.g., meal, may be such that the meal has a lesser value.

Typically in a hexane extraction process the grain is cracked and dehulled and may be provided as a thin flake prior to contact with hexane solvent. Usually several hexane contact stages are used. Hexane is recovered from the oil-laden hexane by fractionation, e.g., using a thin film evaporator, and recycled. Residual hexane is removed from the de-oiled thin flake. This removal is conventionally accomplished by heating the flake and sparging with steam. Especially where the oil or flake is intended for human or animal consumption, the removal of hexane must be relatively complete to avoid any toxicity concerns. As hexane is flammable and can form explosive mixtures, the processing equipment should be explosion proof. Additionally the processing equipment should not discharge hexane either to the atmosphere or in a waste solid or liquid stream due to environmental pollution concerns.

Due to current interest in developing fuels from renewable energy, corn has become a primary source of sugars for ethanol production by fermentation. While food grade corn oil can be extracted from the corn prior to fermentation, the capital costs and additional operating complexities have resulted in many producers using a dry milling process without the recovery of oils. The oils are thus passed through the fermentation and ethanol distillation (beer still) unit operations and are contained in the bottoms from the beer still (whole stillage). In a typical ethanol plant, the whole stillage, which is primarily water with solids (distillers grains), unfermented sugars, and oils, is centrifuged to provide a wet distillers grains fraction and a thin stillage. The thin stillage is processed in an evaporator to remove water which can be reused in the process and a syrup fraction. A commonly adopted option at this point is to recombine the wet distillers grains and the syrup and dry the combination to a moisture content of less than about 11 mass percent to provide a dried distillers grains and solubles (DDGS) that can be sold as an animal feed. The oils, although not toxic, reduce the protein concentration of the DDGS and may not be digestible by certain animals thereby reducing the per unit mass value of the DDGS product.

Jakel, et al., in U.S. Pat. No. 7,083,954 list as solvents for extracting miscible or soluble substances from the cracked grain include hexane, n-hexane, isopropyl alcohol, ethanol and supercritical carbon dioxide.

Cantrell, et al., in US 2006/0041153 propose the recovery of oils from the syrup formed by concentrating the thin stillage by the use of a disk stack centrifuge. They note at paragraph 0006 that attempts to recover oil from the thin stillage before evaporation through the use of centrifuges merely creates an undesirable emulsion phase requiring further processing. See also, US 2006/0041152 of Cantrell, et al.

Winsness, et al., in US 2007/0238891 disclose methods for recovering oil from stillage by heating to separate the oil. The preferred temperature of the heating is at least 212° F. (100° C.) and more preferably the stillage is maintained under superatmospheric pressure to enable heating at between 230° F. and 250° F. The stillage may be hydrolyzed to facilitate removal of the oil. A separator such as a centrifuge or settling tank may be used to recover the oil. One of the uses posed for the removed oil is for biodiesel.

Krasutsky, et al., in US 2007/0089356 disclose extracting oil from distillers grains using solvent. Methanol and ethanol are disclosed as solvents. The extracted oil can be used for the production of biodiesel with low weight alcohol. (Paragraph 0041) At paragraph 0046, Krasutsky, et al., state in discussing FIG. 5 that the ethanol stream can effectively wash wet distillers grains of water and replaces the need for using a hot air stream to dry the wet distillers grains of water. Due to the miscibility of water with these alcohols, it is thus expected that the extract would contain water as well as oil and alcohol, e.g., ethanol.

Cheryan in US 2006/0063920 (which is related to U.S. Pat. No. 6,433,146) uses ethanol to treat dry grind corn. After separating the solids, the ethanol and oil solution is subjected to filtration to provide an oil concentrate and an ethanol permeate. The ethanol is noted by the applicant to absorb water and water must be removed to maintain effectiveness of the ethanol in extracting oil. (Paragraph 0023)

Haas, et al., in US 2006/015538 disclose treating a substrate from which lipids have not been extracted with alcohol and catalyst to form alkyl esters of fatty acids without the expense to recover and purify the lipids. Dried distillers grains and dried distiller grains with solubles are among others stated to be a suitable substrate. They state at paragraph 0084 that extraction of oil costs about 60% of the cost of refined oil and that refined oil is about 75% of the cost of biodiesel.

### SUMMARY OF THE INVENTION

This invention relates to integrated triglyceride-containing oil extraction and biodiesel processes that can provide desirable recoveries of oil without any significant additional safety, health or environmental hazard and without undue energy consumption. By this invention, biodiesel is used to extract oil from biomass derived substrates, or feedstocks. The oil and biodiesel in the extract need not be separated and can be used for making biodiesel under ester forming conditions in the presence of lower alkanol. Biodiesel is currently recognized as substantially non-toxic and is capable of biodegradation. Any biodiesel remaining on or in the substrate from which oil is extracted would thus likely have no undue adverse effect on its subsequent use. Biodiesel's high flash point, which is at least 130° C., and low volatility substantially avoid any risk of fire and explosion.

Further, the extract is susceptible to phase separation from any aqueous phase and contains relatively little dissolved water, usually less than about 0.05 mass percent, and typically at saturation less than about 100 parts per million by mass (ppm-m) water. As water is deleterious to ester formation, either resulting in the formation of free fatty acids and soaps under base-catalyzed transesterification conditions or reduced conversion in acid catalyzed esterification, the processes of this invention provide an extract that can be used to make biodiesel without additional water removal, e.g., by distillation, evaporation, selective sorption or membrane separation.

In the broad aspects, the integrated processes of this invention for the recovery of glycerides from biomass derived feedstock, said feedstock containing glycerides and water, and making biodiesel comprising:
(a) contacting the biomass derived feedstock with a water-immiscible extractant comprising biodiesel under conditions such that at least a portion of the glycerides in the feedstock pass to the extractant to provide an extract containing biodiesel and glycerides and to provide a feedstock having a reduced concentration of glycerides;
(b) phase separating the extract from the feedstock;
(c) subjecting the extract in the presence of lower alkanol to ester forming conditions to convert glycerides to biodiesel and coproduce glycerin; and
(d) recycling a portion of the biodiesel to step (a) as at least a portion of the extractant.

The biomass derived feedstock may be from a plant or animal source and typically contains at least one of water and oil insolubles which may be solid or liquid. Some preferred integrated processes in accordance with this invention are further integrated with the processes for treating the biomass derived feedstock such as processes for making soy meal or other soy products from soybean, processes for making corn meal, and processes for hydrolyzing or fermenting carbohydrates such as corn to ethanol processes. A particularly attractive use of the processes of this invention is with integration with a dry milled corn fermentation process in which oils are contained in the carbohydrate-containing feed to an aqueous fermentation unit operation. The biomass derived feedstock from which the oil is extracted may be one or more of the fermentation medium, or broth, itself, the water-containing fraction after the fermentation product has been removed, and any solids fraction. In an ethanol fermentation process, the biodiesel extractant may contact the whole stillage (including before, during or after any evaporation), the thin stillage (before, during or after evaporation to concentrate the syrup) or the distillers grains.

Where the oil is sought to be removed from the whole stillage or the thin stillage prior to concentration of solids, the use of the biodiesel extractant provides a liquid phase that is often capable of facile separation unlike the emulsion phase that is reported to occur by Cantrell, et al., where only centrifugation is used to remove oil. The removal of oil prior to the separation of the distillers grains or prior to concentrating the thin stillage reduces volume and energy costs especially for concentrating solids and for drying.

The use of biodiesel extractant for removing oil from the fermentation medium can be used to advantage to also recover at least a portion of the alkanol. In broad terms, this one aspect of the process of the invention comprises:
(a) contacting at least a portion of an aqueous fermentation broth containing alkanol, glycerides and water with extractant comprising biodiesel, said contacting being for a time and under conditions including a mass ratio of solvent to broth sufficient to provide an extract containing alkanol and glycerides and an aqueous phase having a reduced concentration of said alkanol and oil;
(b) phase separating the extract and the aqueous phase;
(c) separating by distillation alkanol from the extract to provide an alkanol fraction and an extract fraction containing biodiesel and glycerides;
(d) subjecting at least a portion of the extract fraction in the presence of lower alkanol to ester forming conditions to convert glycerides to biodiesel and coproduce glycerin; and
(e) recycling a portion of the biodiesel to step (a) as at least a portion of the extractant.

In the processes of this aspect of the invention, the biodiesel may be introduced into the fermentation broth during fermentation or a fraction of the fermentation broth can be withdrawn and contacted with the extractant with the aqueous phase being recycled or the fermentation can be completed and the fermentation broth then contacted with the extractant. The contacting can be continuous or intermittent. One mode of intermittent contact operation involves a contacting during the initial phase of the fermentation process which primarily removes glycerides and then one or more contacts later in the fermentation process to primarily remove alkanol and, possible, fermentation inhibitors such as carboxylic acids corresponding to the sought alkanol product. In this preferred mode, the duration that glycerides are subjected to fermentation conditions is reduced as well as subsequent processing unit operations in the fermentation process, thereby reducing degradation of glycerides to, e.g., free fatty acids.

In the preferred processes, the alkanol produced by the fermentation is ethanol, isopropanol, n-propanol, isobutanol and n-butanol, especially ethanol. Where the fermentation broth contains solids, it is preferred to remove at least a portion of the solids before contact with the solvent to remove alkanol to simplify materials handling. It should be recognized that density of the solids will be such that they will be contained in the aqueous phase separated from the solvent, and hence, the solids separation may be conducted or additional solids separation may be conducted by treating the aqueous phase from step (b).

Often the conditions of the contacting in step (a) are sufficient such that the extract contains at least about 20, preferably at least about 30, and most preferably at least about 70, mass percent of said alkanol in the portion of the fermentation broth contacted with the extractant. Frequently at least about 70, and more preferably at least about 90, mass percent of the alkanol produced by the fermentation is removed by contact of the fermentation broth with the extractant.

This aspect of the invention can provide several advantages in addition to reduced energy costs in recovering alkanol by distillation. For instance, with the low solubility of water in biodiesel, the amount of water in the alkanol overhead from the distillation of step (c) may be below the minimum azeotrope amount, e.g., for ethanol, below 5% of the overhead. Thus, as compared to a conventional distillation of ethanol from water, the amount of water that is needed to be removed, e.g., by selective sorption on molecular sieves is less. Hence, the amount of molecular sieve can be reduced per unit of alkanol production or the frequency of regenerations can be reduced. Where the alkanol is removed during the fermentation, a greater production of alkanol by the microorganisms may be achievable as alkanol product is removed to maintain a sufficiently low concentration of alkanol that the alkanol does not inhibit the continued production of alkanol. The extractant can serve to remove fermentation inhibitors such as carboxylic acids. Also, by contacting the fermentation medium with extractant during the fermentation, the time that oils are subjected to fermentation conditions is reduced thereby reducing the extent of any degradation of the glycerides to free fatty acids.

### BRIEF DESCRIPTION OF THE DRAWINGS

FIG. 1 is a schematic depiction of a fermentation process.

FIG. 2 is a schematic depiction of a biodiesel facility useful in the practice of some embodiments of this invention.

FIG. 3 is a schematic depiction of an apparatus for recovering glycerides and alkanol by in situ contact of extractant with fermentation broth in a fermentor.

FIG. 4 is a schematic depiction of an apparatus for recovering glycerides and alkanol by ex situ contact of extractant with fermentation broth.

FIG. 5 is a schematic depiction of an apparatus to remove glycerides from soybeans.

FIG. 6 is a schematic depiction of an apparatus to remove glycerides from whole stillage in an ethanol plant.

### DETAILED DESCRIPTION

In accordance with this invention, biodiesel is used to extract glycerides from biomass derived feedstock. The glyceride-containing extract is subjected to ester-forming conditions in the presence of lower alkanol, thereby generating biodiesel for use as extractant. Thus in the processes of this invention, the glycerides and the biodiesel are not separated in order to provide fresh extractant.

The fresh extractant comprises biodiesel, usually at least about 60 or 65, mass percent biodiesel and may contain a minor amount of glycerides, preferably less than about 30 or 40, and sometimes less than 25, mass percent glycerides. Fresh extractant may be substantially devoid of glycerides, or can contain some glycerides, preferably less than about 25 or 30 mass percent glycerides. The extractant can also contain free fatty acids, but preferably in amounts less than about 10 mass percent. The preferred extractants have a viscosity of less than about 30, preferably less than about 20, millipascal second at 25° C.

### Glyceride Sources

Glycerides are aliphatic glycerides where the aliphatic groups contain between about 8 and 30, often between about 16 and 20 or 24 carbon atoms, for most biomass sources of glycerides. Triglycerides have three such aliphatic groups, diglycerides, two such groups, and monoglycerides, one such group. The glyceride-containing biomass for the feedstock are from biosources, especially vegetables and animal fats. Examples of glyceride-containing biomass include, but are not limited to rape seed, soybean, cotton seed, safflower seed, castor bean, olive, coconut, palm, corn, canola, jatropha, rice bran, tobacco seed, fats and oils from animals, including from rendering plants and fish oils.

The biosource may be used as obtained from the plant or animal but is generally treated to enhance the ability of the extractant to remove oils. The treatment may comprise one or both of physical and chemical treatment. For instance, the biosource may be ground or flaked. Plant sources such as seeds may be dehulled, and with respect to animal fats, non-fat components such as meat and sinew removed. The biomass derived may be another product or intermediate using the biosource such as bone meal and distillers grains. Chemical treatments include hydrolysis and enzymatic and microbiological digestion, and the like. The chemical treatments may affect the non-oil components of the biosource. Generally, such chemical treatments are selected to minimize the degradation of the oils, e.g., to free fatty acids.

Thus, the processes of this invention can be used to obtain glycerides from animal and plant-derived sources such as ground, crushed, flaked, or otherwise physically processed biomass. For instance, the processes of this invention can be used to extract glycerides from crushed, ground, flaked or otherwise physically processed soybean, palm, castor bean, rape seed, cotton seed, olive, coconut, corn, canola, jatropha, rice bran, tobacco seed, animal fat, bone meal and the like.

The processes of this invention can also be used to recover glycerides from processes in which the biomass is being chemically processed to obtain other useful materials. Particularly attractive processes for the practice of the processes of this invention include fermentation processes such as those used to produce alkanols and carboxylic acids. Alkanols, including dialkanols that can be produced by fermentation of carbohydrates have 1 to 5, especially 1 to 3, carbon atoms and include methanol, ethanol, propanol, isopropanol, propanediol, butanol, isobutanol, and the like. Carboxylic acids, such as can be produced by homoacidogenic fermentation include monoacids and diacids, especially those having from 1 to 5, especially one to 3, carbon atoms. The acids may or may not be substituted, e.g., with hydroxyl or lower alkoxy moieties. Exemplary acids include, but are not limited to formic acid, acetylformic acid, acetic acid, hydroxyacetic acid, methoxyacetic acid, propionic acid, hydroxypropionic acid, and butyric acid.

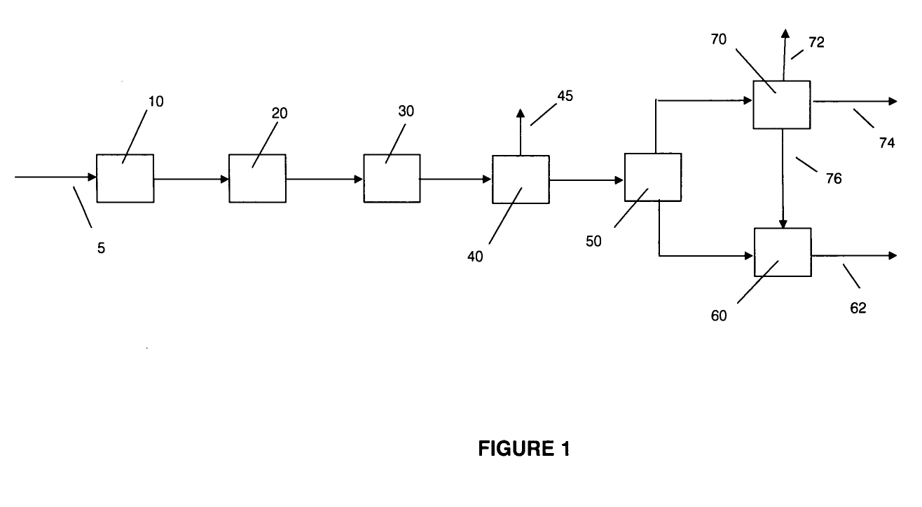

For purposes of assisting in understanding fermentation processes, FIG. 1 provides an overview an ethanol plant. It should be understood that similar processes can be used for fermentation of carbohydrates to other chemicals.

Any suitable carbohydrate-containing feedstock can be used for the fermentation that is converted to the sought product by the chosen microorganism. Carbohydrates are compounds containing carbon, oxygen and hydrogen that contain a saccharose unit or its first reaction product and in which the ratio of hydrogen to oxygen is the same as in water. Any suitable carbohydrate-containing feedstock may be used in the processes of this invention that is converted to acetic acid by the chosen microorganism for the fermentation. Examples of carbohydrate-containing feedstocks are cellulosic materials such as derived from wood, grasses, cotton, corn stover, and the like, especially hemicellulosic materials; starches and sugars including, but not limited to, xylose, sucrose, dextrose, fructose, lactose, maltose, cellobiose, gum Arabic, tragacanth, and the like. The sugars may be derived from various sources such as sugar cane, sugar beet, milk, milo, grapes, sorghum, maple syrup, corn, and the like. The carbohydrate-containing feedstocks may be used directly, but most often are pretreated to recover other useful components therefrom or to convert the carbohydrate into a form more suitable for fermentation. Examples of pretreatment include milling; extraction; enzymatic hydrolysis and chemical treatment such as hydrolysis. Particularly advantageous sources of carbohydrate-containing feedstocks are sugar cane, sugar beets, wheat and corn.

For purposes of discussion, corn will be the carbohydrate source for the fermentation and ethanol the product.

Corn is provided by line 5 to milling operation 10 where the corn is dehulled and milled. Either wet or dry milling may be used to provide a corn meal or flaked corn product. The meal or flaked product contains carbohydrates and is passed to hydrolyzer 20 where by actions of chemicals or enzymes a hydrolyzate is produced. In the hydrolyzer the carbohydrates are converted to sugars desirable for fermentation.

The hydrolyzate is passed to fermentor 30 where microorganisms consume sugars and other carbohydrates to make the fermentation product. The fermentors may operate in a batch, semi-continuous or continuous mode. The conditions of the fermentation can fall within a broad range depending upon the microorganism used, the sought product, and the fermentor design. The fermentation may be continuous, semi-continuous or batch. In some instances, the fermentors are shut down and cleaned before any deleterious mutation of the microorganism can occur.

The fermentation is conducted in an aqueous medium. Generally, the concentration of carbohydrate to water is in the range of about 2 to 50, preferably 3 to 20, and most often between about 3 and 10, mass percent. Amino acids and trace metals and other components may need to be provided, if not contained in the feedstock, to assure a sufficient nutrient medium for the microorganisms. Buffers may also be present. The temperature of the fermentation is often within the range of about 25° to 75° C., say, about 40° to 70° C. The fermentation may be conducted in batch or continuous or semi-continuous modes. Advantageously, the fermentation vessel is agitated, e.g., by stirring, pumped recycle or vibration. The microorganism may be dispersed in the fermentation menstruum or growing on a solid support such as activated carbon, pumice stone and corncob granules. The fermentation may occur in a single stage, or two or more sequential fermentation stages may be used.

The fermentation may be aerobic or anaerobic fermentation and is conducted in an aqueous menstruum in the presence of nutrients and growth factors for the microorganism. Any suitable microorganism capable of producing the sought product can be used. Numerous microorganisms are known for the generation of alkanols. Representative microorganisms include Sacchromyces such as Sacchromyces cerevisiae, Zygosaccharomyces, and Brettanomyces. Often used microorganisms for making alkanols co-produce carbon dioxide and the fermentation is under anaerobic fermentation conditions.

Numerous microorganisms are known for homoacidogenic fermentation. Representative acidogenic microorganisms are those of the Acetobacterium, Clostridium, Lactobacillius, and Peptostreptococcus species, such as Clostridium thermoaceticum, Acetogenium kivui, Acetobacterium woodii, Clostridium formicoaceticum, Lactobacillius casei, Lactobacillius delbruckii, Lactobacillius heiveticus, Lactobacillius acidophilus, Lactobacillius amylovorus, Lactobacillius leichmanii, Lactobacillius bulgaricus. Lactobacillius amylovorus, Lactobacillius pentosus, Propionibacterium shermanii, Clostridium butyricu, Clostridium tyrobutylicum, Propionibacterium acidipropionic, and Clostridium thermobutyricum. Anaerobic conditions are usually used for homoacidogenic fermentation and little, if any, carbon dioxide is produced.

In most fermentation processes, as the concentration of the sought fermentation product increases in the fermentation medium, the rate of production slows and the microorganism may be inhibited or inactivated. The maximum concentration will be dependent upon the microorganism and the sought product. For example, yeasts are available that enable an ethanol concentration in the fermentation medium in excess of 10 percent. In the production of acetic acid, the maximum concentration of acid may be less than about 5 or 8 percent. Often the fermentation produces fermentation inhibitors. For example, acetic acid can be produced during fermentation of sugars to provide ethanol, and the acetic acid can act as a fermentation inhibitor.

One of the advantages of the aspects of this invention where sought product is removed by the extractant during the fermentation, is the ability to maintain the concentration of the sought fermentation product and any oil-miscible inhibitors below that which unduly adversely affect the microorganism's production rate.

The fermentation broth, or beer, is then fractionated in distillation assembly 40 to provide the sought fermentation product via line 45. The distillation assembly may also include dryers, e.g., molecular sieve dryers, to remove any water remaining in the fermentation product. For example, the distillation may produce an azeotrope of ethanol and water and the dryers can remove the water to provide essentially pure ethanol. Alternatively extractive distillation with aromatic or aliphatic hydrocarbon can be used to remove water from the azeotrope.

The bottoms, or whole stillage, from distillation assembly 40 is passed to centrifuge 50 to provide a solids fraction, distillers grains, which is passed to dryer 60. The liquid from centrifuge 50, or thin stillage, is passed to evaporator 70 to provide a water overhead exiting via line 72 which can be recycled for use in the process and a concentrate, or syrup. Typically the evaporator comprises a number of stages. The syrup may be withdrawn as a by-product via line 74. All or a portion of the syrup may be passed via line 76 and added to the distillers grains and dried in dryer 60. Dryer 60 provides via line 62 dry distillers grains or dry distillers grains with solubles if all or a portion of the syrup is recombined with the distillers grains. Alternatively, the whole stillage including distillers grains can be subjected to water removal by evaporation and then drying to provide a distillers dried grains product.

The processes of this invention can be applied at one or more of the sections depicted in FIG. 1. For example, extraction with biodiesel can occur during or after physical processing in milling operation 10; during or after hydrolysis in hydrolyzer 20; during or after fermentation in fermentor 30; in lieu of or after distillation in distillation assembly 40; by treatment of distillers grains after centrifuging in centrifuge 50 including before or after drying and before, during or after forming distillers dried grains with solubles; by treatment of the thin stillage after centrifuging; and during or after evaporation of the thin stillage in evaporator 70.

Distillers dried grains can be used for fuel or, more significantly, animal feed and feed supplements. In one embodiment, the processes of this invention are used to not only extract oils from distillers dried grains or distillers dried grains with solubles, but also, the extraction is conducted at a temperature sufficient to substantially sterilize the distillers dried grains, e.g., at least about 80° C., say, 85° C. to 120° C., where the temperature is maintained for at least about 30 seconds, preferably, between about 1 and 60 minutes.

### Extraction and Separation

The contacting of the extractant containing biodiesel and the biomass derived source of glycerides is under conditions suitable for extracting at least a portion of the glycerides. Preferably at least about 50, more preferably at least about 60, and sometimes at least about 90, mass percent of the glycerides are extracted upon completion of the extraction.

Frequently the temperature of contact is between about 10° C. and 120° C., preferably between about 25° C. and 100° C. or 110° C., although higher temperatures, e.g., up to about 150° C. can be used due to the high flash point of the extractant. Higher temperatures reduce the viscosity of the extractant and thus facilitate dispersion of extractant in solid or semi-solid biomass derived sources of glycerides. The dispersion aids in the removal of the glycerides. Where the biomass derived source is not deleteriously affected by higher temperatures, extraction temperatures in excess of 50° C., say, in excess of 60° C. or preferably in excess of 70° C., may be desirable to provide the extractant with a viscosity less than about 5, most preferably less than about 2, millipascal second. Examples of such biomass derived sources include the whole stillage, the thin stillage, or the distillers grain. Higher temperatures may be preferred where the biomass derived source of glyceride is solid or of high viscosity at lower temperature. Where the biomass derived source is a fermentation medium, lower temperatures may be preferred to avoid damage to the microorganism. Often the contacting is done at a temperature where the solvent has a viscosity of less than about 20, preferably less than about 10, and often less than about 5, millipascal second.

The solvent may comprise the continuous phase or may be discontinuous during the contacting with a liquid phase. Where discontinuous, the droplet size of the solvent phase may be up to about 5 centimeters in diameter, but usually is in the range of from about 0.5 to 2500, preferably 50 to 2000, microns in diameter. Usually the contacting is at the pressure of the fluids being passed to the contacting zone, e.g., from about 90 to 500 kPa (absolute) although higher or lower pressures can be used without undue adverse effect.

During milling, typically a dry corn flour is produced. In one embodiment of this invention, the corn, milled or undergoing milling, may be contacted with the solvent. By effecting the milling or contacting with solvent prior to any addition of water, any hydrolysis of glycerides to free fatty acids prior to extraction is minimized. Alternatively, the milled corn may be first contacted with water and then with solvent to extract glycerides. In either case, the spent extractant is removed from the solids, or water and solids, as the case may be. The separation may be effected by any suitable method, including but not limited to phase separation by settling, filtration, or centrifugation.

Where the solvent is contacted with dry corn and separated, typically the solids separated from the extractant are wetted with spent extractant and thus are in the form of a wetted mass. Water may be used to slurry the mass to facilitate transport to subsequent unit operations. If desired, a subsequent separation may be used to remove residual spent extractant from the solids. Alternatively, the solvent and solids composite may be contacted with water prior to separation of the spent extractant.

One of the advantages of this mode of extraction of glycerides is that corn that is not capable of being dry milled, such as high oil content corn, can be used.

Where the contacting is done in situ or ex situ during a hydrolysis or fermentation, the contact between the hydrolysis broth or fermentation broth and the extractant may be continuous or intermittent. Where the hydrolysis or fermentation is conducted in a batch or semi-batch mode, the contacting and relative volume of solvent used can vary as the hydrolysis or fermentation progresses. By way of example, the contacting may not occur until a desired amount of alkanol has been produced in the fermentation. The rate of introduction of solvent may increase or decrease with an increase or decrease in rate of production of the alkanol during a fermentation. It may be desirable to have an initial contact to remove glycerides.

The volume ratio of extractant to biomass derived feedstock will depend upon not only the portion and absolute amount of glycerides to be removed, but also the apparatus used. Considerable flexibility exists in the volume ratio of solvent to source without unduly adversely affecting the process economics. For instance, where glycerides are to be extracted from solids or semi-solids, sufficient extractant may be used to form a slurry. Alternatively, extractant may be passed through a fixed bed of solids. At any given time, the total mass of extractant to mass of solids or semi-solids is typically in the range of about 1:1 to 20:1, say, 2:1 to 10:1.

For biomass derived feedstocks that are liquid, generally lesser volumes of extractant are required due to the enhanced ability to disperse the extractant and the glyceride-containing liquid. Slurries are considered to be liquids for the purposes of this discussion. Generally the volume ratio of extractant to glyceride containing liquid is in the range of about 0.05:1 or 0.1:1 to 5:1. Where the contacting is done in, for instance, a fermentor, the ratio is typically in the range of about 0.1:1 to 0.6:1. Otherwise the volume ratio is usually in the range of about 0.2:1 to 5:1, preferably 0.5:1 to 3:1. If an emulsion tends to be formed during phase separation of the extractant and the source, the use of higher ratios of extractant may attenuate emulsion formation.

The extraction can be continuous, semi-continuous (intermittent) or batch. The residence time of the contact between the extractant and the feedstock is generally less than about 5 hours, frequently less than about 30 minutes, preferably between about 10 or 30 seconds and 5 or 10 minutes. During the period of contacting, sufficient extractant is provided such that a mass ratio of glycerides to extractant is in the range of between about 0.01:1 to 1:1, preferably between about 0.05:1 to 0.5:1. In some instances the extract from the contacting contains at least about 20, preferably at least about 30, mass percent glycerides based upon the total mass of the extract.

Any suitable apparatus can be used for the contacting. Preferably for liquid-liquid systems the contacting is done in a countercurrent mode which may be effected in a single vessel, e.g., countercurrent extraction vessel which may or may not be agitated or contain contact surfaces to enhance phase contact, or in a series of separate vessels which may or may not be agitated or contain contact surfaces to enhance phase contact. For systems involving solids containing glycerides, the contacting can be in vessel containing a slurry of the solids or in a moving or fixed bed of solids through which extractant is passed.

The aqueous and solvent phases are subjected to phase separation. This phase separation may be inherent in the apparatus used for the contacting or may be subsequent to the contacting step. For solids, the phase separation may be by settling, centrifugation, filtration or the like. For liquid-liquid separations, decanting, centrifugation and the like may be useful. Where solids or semi-solids are being subjected to the extraction, residual extractant may reside in the interstices among the solids or within the structure of the solids, the use of water or steam to remove residual extractant is an optional unit operation. The water and extractant can then be phase separated.

### Biodiesel Production and Extractant Regeneration

The source of the biodiesel for the extractant and the regeneration of the extractant is through subjecting the spent extractant to ester forming conditions in the presence of lower alkanol. The ester forming conditions can be acid catalyzed esterification and base catalyzed transesterification. As the regenerated extractant can contain some glycerides, it is not essential that the esterification be sufficient to react all the glycerides. The conversion of glycerides to biodiesel should, however, be adequate to assure steady state operation for a continuous process. Where, however, the objective is to make biodiesel, it is preferred that substantially all the glycerides are esterified, even though an intermediate product containing glycerides is used as the extractant.

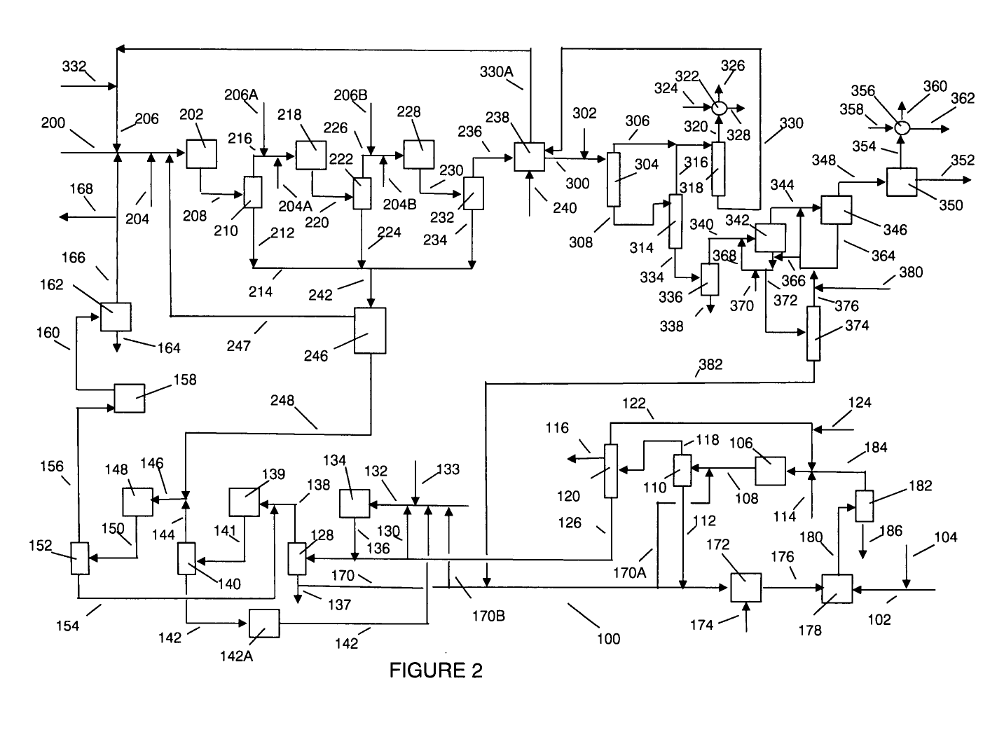

For purposes of discussion reference will be made to the biodiesel facility of FIG. 2. It should be kept in mind that any of the acid-catalyzed esterification section or the pretreatment section or the transesterification section could be used solely for the regeneration of extractant. Such an intermediate product containing biodiesel and partially reacted glycerides can be valuable not only as extractant but also as a saleable product as a feed to other biodiesel facilities. Hence, it is not essential in the broad aspects of the invention that biodiesel be made as a product in the extractant regeneration.

FIG. 2 schematically depicts biodiesel manufacturing facility 100. Facility 100 is provided with a transesterification component (generally designated by numerals in the 200 series) as well as pretreatment components (generally designated by numerals in the 100 series) and a refining component generally (designated by numerals in the 300 series).

### Pretreatment by Esterification

As shown in FIG. 2, a glyceride feed containing free fatty acid can be provided to facility 100 via line 102 for pretreatment by acid. Line 104 is provided in the event that more than one feed is desired to be processed simultaneously in the esterification section. Catalyst, which for purposes of this discussion, is sulfuric acid, is provided via line 114.

The feed may be directly introduced into esterification reactor 106, or as shown, is subjected to a contact with an alkanol laden stream of glycerin to strip alkanol from the glycerin into the oil-containing feed phase. This contact will be described later.

The preferred conditions for the esterification will depend upon the nature of the feed and the apparatus type and configuration. Reactor 106 may comprise one or more stages or vessels and separation unit operations may be located between each stage or vessel. Where reactor 106 is staged, it is often desirable, but not essential, to remove water between stages to enhance conversion of free fatty acid to esters. Reactor 106 may be a vessel or a length of pipe. But preferably other types of vessels are used such as mechanical and sonically agitated reactors, and reactors with static mixing such as reactors containing contact structures such as trays, packing, baffles, orifices, venturi nozzles, tortuous flow path, and other impingement structures. Suitable reactors include those providing high intensity mixing, including high shear.

The oil phase from the esterification section of facility 100 often contains at least about 0.5, say between about 0.5 and 2 or 3, mass percent free fatty acid. This free fatty acid serves to neutralize at least a portion of the base catalyst contained in a spent glycerin stream produced in the transesterification and base pretreatment sections of facility 100. Preferably, the molar ratio of free fatty acid in the oil phase from the esterification to mole of base in the glycerin phase introduced into base reactor 134 as discussed below will be at least about 0.3:1, often at least about 0.7:1 up to about 1:1. The use of ratios of free fatty acid to base catalyst of greater than 1:1 can adversely affect the performance of the base pretreatment. A number of advantages flow from this preferred embodiment. For instance, the equipment and conditions required for the esterification section need not be of the type required for essentially complete conversion of the free fatty acids, resulting in capital and operating cost savings. Since residual free fatty acid is converted to soap and removed in the base pretreatment section, the feed to the transesterification section can be substantially devoid of free fatty acid which adversely affects the base catalyst therein. Additionally, the neutralized spent glycerin stream from the base pretreatment section can be used effectively for enhancing phase separation and water and catalyst removal from the esterification product.

During the esterification in reactor 106 some conversion of glycerides to esters may occur. The esters, diglycerides and monoglycerides essentially remain in the oil phase. Some glycerin will be produced as a result of the transesterification of the glycerides in the feed. The extent of such conversion is not critical but results in reduced requirements of alkanol and catalyst in the transesterification section per unit of biodiesel produced as well as enabling increased performance such as rate of conversion and extent of conversion to be obtained. Generally up to about 20 mass percent, say, between about 0.1 to 15, and sometimes between 5 to 10, mass percent of the glyceride-containing feed is transesterified during acid esterification.

The esterification reaction product from reactor 106 is passed via line 108 to phase separator 110. Phase separator 110 is optional depending upon whether or not two phases exist. In some instances, an oil layer containing glycerides and fatty ester and a water-containing layer form. The water-containing layer can contain more polar components such as glycerin, water-soluble catalyst, and alkanol. As shown, a neutralized spent glycerin stream from the base pretreatment section is provided via line 170A and contacted with the esterification product. The spent glycerin aids in the extraction of water and water-soluble phosphorus compounds. Additionally, the glycerin assists in making the phase separation. In this embodiment, the amount of glycerin added can vary widely. As relatively small amounts of water are produced during the acid esterification of free fatty acids, beneficial results can be achieved with relatively little spent glycerin being added. Often the spent glycerin added is less than about 20, preferably between about 0.5 and 10, mass percent of the stream from esterification reactor 106. A separate phase may exist in reactor 106, e.g., from catalyst such as sulfuric acid, or water co-produced during the esterification or even alkanol above that miscible with the oil phase. Glycerin can aid in forming a defined phase containing, e.g., catalyst and water. As used herein, the formation of a glycerin phase or providing a glycerin phase contemplates that there may, or may not, be separate phases in the fluid contacted with glycerin. Spent glycerin that is in a separate phase may be separated and removed via line 112.

Phase separator 110 may be of any suitable design including a decanter, a phase separation facilitated decanter that contains coalescing sites, and a centrifuge. The lower, water-containing fraction exits separator 110 via line 112. This fraction contains some alkanol, water, water-soluble catalyst, glycerin and water-soluble phosphorus compounds.

The oil fraction of separator 110 contains virtually no sulfuric acid, often some alkanol, relatively little water, unreacted free fatty acids, if any, fatty ester and glycerides. The fraction is passed via line 118 from separator 110 to fractionation column 120 to provide an overhead fraction containing alkanol and a bottoms stream containing oil. The overhead from column 120 can be recycled to esterification reactor 106 via line 122. Make up alkanol is provided via line 124.

The fractionation column may be of any suitable design including a flash column, stripping column, falling film evaporator, or trayed or packed column. If desired, more than one fractionation column can be used with one effecting separation of water from alkanol. Similarly a side draw 116 may be taken from distillation column 120 for the removal of water, and fractionation column may be a divided wall column to enhance such separation. In an embodiment, a substantial portion of the water is removed by the phase separation in phase separator 110, and fractionation column does not separately recover water. Water will be contained in both the overhead and bottoms stream from column 120. However, the relatively small amount of water in the overhead can be recycled with alkanol via line 122 to reactor 106 without undue adverse effect. Water contained in the bottoms passes to the base pretreatment section and is removed from the oil phase therein.

In another embodiment, only a portion of the alkanol is removed by fractionation in column 120. The alkanol remaining in the oil phase is passed to the base pretreatment section. In the base pretreatment section alkanol can be reacted with glyceride to form esters and can be recovered in the spent glycerin phase for recycle to the esterification section. Thus, the capital and operating costs for fractionation column 120 can be reduced. Often the bottoms stream from fractionation column 120 contains between about 0.1 to 10, say, between about 0.5 and 5, e.g., 0.5 to 2, mass percent alkanol. In yet another embodiment, the oil-containing fraction from separator 110 can be passed directly to separator 128 or base reactor 134.

While shown as processing the oil phase from separator 110, fractionation column 120 may be positioned between esterification reactor 106 and separator 110 and serve to recover alkanol from the esterification product exiting reactor 106.

Pretreatment by Base

The base pretreatment uses glycerin produced in facility 100 to treat feed. The base pretreatment serves to recover alkanol contained in the glycerin phase from the transesterification section. Hence, the spent glycerin from the base pretreatment section may contain relatively little alkanol. Base pretreatment also serves to partially convert glycerides in the feed to fatty acid esters and mono- and di-glycerides. Thus, the amount of alkanol required to transesterify the pretreated feed will be less than had no base pretreatment occurred. Base pretreatment can also serve to remove phospholipids as glycerin-soluble components. Base pretreatment further removes free fatty acids from the glyceride-containing feed by saponification to glycerin-soluble soaps. Removal of the phospholipids and free fatty acids facilitates processing during transesterification and minimizes catalyst loss during transesterification cased by saponification of free fatty acids with base catalyst. Phospholipids, for instance, tend to make more difficult phase separations of oil and glycerin in the transesterification component. And biodiesel must meet stringent phosphorus specifications. See, for instance, ASTM D 6751, American Society for Testing and Materials.

As shown in the facility of FIG. 2, a glyceride-containing feed stream is provided by line 132 to base reactor 134. The feed stream may comprise a fresh glyceride-containing feed. Alternatively or in addition, the feed stream may comprise the oil phase from the esterification provided via lines 126 and 130. To base reactor is also provided a glycerin and base catalyst-containing stream via line 142 which will be further discussed below. Preferably a non-acidic inerting gas such as nitrogen or hydrocarbon gas such as methane is used during base pretreatment.

In base reactor 134, free fatty acids contained in the feed stream are reacted with base catalyst to form soaps. If the free fatty acid content of the feed stream requires more than the amount of base catalyst introduced via line 142 for the desired degree of saponification, additional base can be added via line 133. The additional base may be the same or different from that comprising the catalyst, and may be one or more of alkali metal hydroxides or alkoxides and alkaline earth metal hydroxides, oxides or alkoxides, including by way of examples and not in limitation, sodium hydroxide, sodium methoxide, potassium hydroxide, potassium methoxide, calcium hydroxide, calcium oxide and calcium methoxide.

To the extent that phospholipids are present in the feed stream to base reactor 134, at least a portion is chemically reacted, e.g., by a hydration or by a salt formation, to provide chemical compounds preferentially soluble in glycerin.

Base reactor 134 is maintained under base reaction conditions, which for free fatty acid-containing feed streams is that sufficient to react basic catalyst and free fatty acids to soaps and water, and for phospholipids-containing feed streams is that sufficient to react basic catalyst and phospholipids to chemical compounds preferentially soluble in a glycerin phase. Typical base reaction conditions include a temperature of at least about 10° C., say, 35° C. to 150° C., and most frequently between about 40° C. and 80° C. Pressure is not critical and subatmospheric, atmospheric and super atmospheric pressures may be used, e.g., between about 1 and 5000, preferably from about 90 to 1000, kPa absolute. The residence time is sufficient to provide the sought degree of saponification of fatty free acids and reaction of phospholipids. The residence time in base reactor 134 may range from about 1 minute to 10 hours.

Base reactor 134 may be of any suitable design. Reactor 134 may be a vessel or a length of pipe. But preferably other types of vessels are used such as mechanical and sonically agitated reactors, and reactors with static mixing such as reactors containing contact structures such as trays, packing, baffles, orifices, venturi nozzles, tortuous flow path, and other impingement structures. Suitable reactors include those providing high intensity mixing, including high shear.

The base reaction product from reactor 134 contains glycerin, glycerides, soaps, water, and fatty acid ester and is passed via line 136 to separator 128. Separator 128 serves to separate the less dense oil layer from the more dense glycerin layer. The soaps and reacted phospholipids preferentially pass to the glycerin layer as does most of the water. The oil layer preferably contains less than about 0.5 mass percent soaps. Phase separator 128 may be of any suitable design including a decanter, a phase separation facilitated decanter that contains coalescing sites, and, if needed, a centrifuge.

The glycerin phase is withdrawn from separator 128 via line 137 and may be sent to glycerin recovery or another application. If the glycerin layer contains significant amounts of soaps, it may be desirable to recycle the soaps to esterification reactor 106 for conversion to fatty esters. As shown, a portion or all of the glycerin phase may be passed via line 170 to acidification reactor 172 where soaps are converted to free fatty acids. At least a portion of this glycerin phase is passed via line 170A to provide the glycerin to assist in the separation of water, water-soluble catalyst (or salts thereof) from the esterification product in phase separator 110. The glycerin-containing phase from separator 110 is passed via line 112 to line 170. Also as shown, a portion of the glycerin phase in line 172 is recycled to reactor 134 via line 170B. The recycle can serve several purposes. For instance, hydrated phospholipids are returned to reactor 134 where they may undergo transesterification to recover additional fatty acid ester. Also, any base contained in the recycled glycerin stream is available for saponification of free fatty acids.

Unless acid contained in the esterification effluent of line 108 is neutralized prior to being passed to separator 110, the glycerin-containing phase from separator 110 will contain water-soluble acid which can be used as acid for acidification reactor 172. Acid can also be provided via line 174. Acidification reactor 174 may be one or more vessels of any suitable design including a length of pipe and other types of vessels such as mechanical and sonically agitated reactors, and reactors with static mixing such as reactors containing contact structures such as trays, packing, baffles, orifices, venturi nozzles, tortuous flow path, and other impingement structures. Suitable reactors include those providing high intensity mixing, including high shear. The acidification conditions usually encompass a temperature in the range of about 20° C. to 150° C., a pressure from about 1 to 5000, preferably 90 to 1000, kPa absolute, and a residence time of from about 1 second to 5 hours. Suitable acids include mineral acids and organic acids, but typically a readily available acid such as sulfuric or phosphoric acid is used. The amount of acid is usually sufficient to convert substantially all the soaps to free fatty acid. The use of excess acid is not deleterious to the formation of the free fatty acids, but can entail additional expense. Accordingly the molar ratio of acidifying acid function to soaps is in the range of about 1:1 to 1.5:1. Generally the pH of the glycerin stream is less than about 6, say, between about 1 and 5, e.g., 2 and 4. The acidity of the glycerin stream is determined by diluting the glycerin stream to 50 volume percent water and measuring the pH.

The glycerin stream from acidification reactor is passed via line 176 to contact vessel 178 into which glyceride-containing feed is provided via line 102. In contact vessel 178 the glycerin stream is contacted with fresh feed which serves to extract a portion of the alkanol from the glycerin phase. The contact with the glycerin also serves to remove water from the feed. Removal of water assists in the esterification of free fatty acids in esterification reactor 106 as the esterification is an equilibrium-limited reaction affected by water concentration.

Contact vessel 178 may be of any suitable design including a length of pipe and other types of vessels such as mechanical and sonically agitated reactors, and reactors with static mixing such as reactors containing contact structures such as trays, packing, baffles, orifices, venturi nozzles, tortuous flow path, and other impingement structures. Suitable reactors include those providing high intensity mixing, including high shear. The contact conditions usually encompass a temperature in the range of about 20° C. to 150° C., a pressure from about 1 to 5000 kPa absolute, and a residence time of from about 1 second to 5 hours. Often at least about 50 mass percent of the alkanol in the glycerin stream passes to the oil phase as do essentially all of the free fatty acids. The amount of alkanol recovered from the glycerin will depend upon the alkanol content of the glycerin, the ratio of glycerin to fresh feed, and the contacting conditions. Frequently the mass ratio of glycerin to oil is in the range of between about 1:5 to 1:20, say 1:8 to 1:15, and at least about 30, and sometimes between about 50 and 99, mass percent of the alkanol in the glycerin phase passes to the oil phase.

The ability to recover alkanol from glycerin by extraction with fresh feed can effectively be used to use glycerin as a complementary means for recycling unreacted alkanol to reactor 106. FIG. 2 shows two glycerin loops for alkanol recovery and recycle to the esterification reactor. The first loop involves the glycerin layer from separator 110 and the second, the glycerin layer from separator 128.

The fluid mixture from contact vessel 178 is passed via line 180 to phase separator 182. In phase separator 182, a glyceride and free fatty acid oil layer is produced that is passed via line 184 to esterification reactor 106. A glycerin-containing layer is discharged via line 186 and contains water, acidification acid, and soluble phosphorus compound. Separator 182 may be of any suitable design including a decanter, a phase separation facilitated decanter that contains coalescing sites, and, if necessary, a centrifuge. Contact vessel 178 and decanter 182 may be a single vessel, including but not limited to, a countercurrent extraction column.

If the esterification product from esterification reactor 106 has a sufficiently low free fatty acid content and low phospholipids content, another option is to eliminate separator 110 and fractionation column 120 and provide the esterification product in line 108 directly to separator 128 or base reactor 134.

Returning to separator 128, the oil phase is withdrawn and passed via line 138 to second pretreatment reactor 139. Second pretreatment reactor 139 and third pretreatment reactor 148 are adapted to recover alkanol contained in the glycerin from the transesterification component of facility 100 through reaction, e.g., transesterification and extraction into the glyceride-containing phase. A base transesterification process is used in these pretreatment reactors. While two reactors are shown, the number of reactors will depend upon the sought consumption of the alkanol as well as the efficiency of the reactors. Hence one, two, or three or more pretreatment reactors may be used. Also, the pretreatment reactor can comprise a number of stages in a single vessel which could be a countercurrent contact vessel. Advantageously, the feed stream to the alkanol consumption pretreatment reactors is relatively free from free fatty acids so as to prevent undue consumption of the base catalyst. Typically the pretreatment reactors provide a glycerin stream from which most of the alkanol has been removed. Often, the alkanol content of the glycerin discharged from base reactor 134 is less than about 5, and preferably less than about 2, mass percent.

In an alternative mode of operation, a significant portion of the alkanol is contained in line 126 (or line 108 if separator 110 and distillation column 120 are not used) and passed to separator 128. The concentration of alkanol in the glycerin-containing stream in line 170 may be higher than 5 mass percent, and alkanol is recovered be partitioning to the glyceride-containing feed in contact vessel 178. The alkanol content of the glycerin may be sufficiently low that no distillation is required to recover alkanol yet the overall process to make biodiesel can still exhibit high efficiencies.

Second pretreatment reactor 139 also receives the glycerin phase from the third pretreatment reactor. This glycerin phase contains glycerin, base catalyst, and alkanol. Second pretreatment reactor 139 is maintained under base transesterification conditions including the presence of base catalyst provided by the glycerin phase feed and elevated temperatures, often between about 30° C. and 220° C., preferably between about 30° C. and 80° C. to provide a second pretreatment product. The pressure is typically in the range of between about 90 to 1000 kPa (absolute) although higher and lower pressures can be used. The reactor is typically batch, semi-batch, plug flow or continuous flow tank. Preferably other types of vessels are used such as mechanical and sonically agitated reactors, and reactors with static mixing such as reactors containing contact structures such as trays, packing, baffles, orifices, venturi nozzles, tortuous flow path, and other impingement structures. Suitable reactors include those providing high intensity mixing, including high shear. However, depending upon the presence of soaps and phospholipids, care needs to be taken so as not to generate a product that cannot be readily separated by phase separation. The residence time will depend upon the desired degree of conversion of the contained alkanol, the ratio of alkanol to glyceride, reaction temperature, the degree of agitation and the like, and is often in the range of about 0.1 to 20, say, 0.5 to 10, hours.

The second pretreatment product contains glycerides, fatty esters, base catalyst and glycerin, and it has a reduced concentration of alkanol. The second pretreatment product is passed from second pretreatment reactor 139 via line 141 to separator 140. Separator 140 may be of any suitable design including a decanter, a phase separation facilitated decanter that contains coalescing sites, and, optionally, a centrifuge. The lower, glycerin-containing phase from separator 140 contains relatively little alkanol, preferably less than about 10 mass percent, and contains base catalyst, and is passed via line 142 to base reactor 134 where catalyst reacts with free fatty acids to form soaps which can then be removed from the glyceride-containing feed.

As depicted, line 142 is provided with holding tank 142A. Holding tank 142A can serve as a reservoir and enables the rate that glycerin, which contains base, is provided to base reactor 134, to be varied with changes in free fatty acid content of the esterification product. It also can permit additional reaction of glycerides with alkanol contained in the glycerin phase to occur prior to introduction into base reactor 134 where catalyst is consumed by conversion of free fatty acids to soaps.

The upper oil phase is removed from separator 140 via line 144 and is passed to line 146 which also receives the glycerin co-product from transesterification from line 248. The combined streams are passed to third pretreatment reactor 148. The stream is provided by line 146 and contains in addition to glycerin, alkanol, base catalyst, and usually some water and soaps. Table I sets forth typical compositions of the stream in line 248. The compositions, of course, will depend upon the operation of the transesterification component as well as which of the glycerin-containing streams from the transesterification component are used. The typical concentrations are based upon combining all glycerin-containing streams and operating under preferred parameters.

TABLE I

| Component | Broad, Mass % | Typical, Mass % |
|---|---|---|
| Glycerin | 40 to 80 | 50 to 70 |
| Alkanol (Methanol) | 15 to 50 | 25 to 45 |
| Catalyst (NaOCH₃) | 0.2 to 5 | 0.5 to 5 |
| Soaps | 0.1 to 5 | 0.5 to 5 |
| Water | 0.01 to 0.5 | 0.05 to 0.3 |
| Oil (glycerides and alkyl | 0 to 5 | 0.5 to 2 |
| esters) | | |

Third pretreatment reactor 148 is maintained under base transesterification conditions including the presence of base catalyst provided by the glycerin-containing feed and elevated temperatures, often between about 30° C. and 220° C., preferably between about 30° C. and 80° C. to provide a first pretreatment product. Base catalyst in the transesterification component tends to partition to the glycerin phase and often adequate catalyst is provided for the base pretreatment section in the glycerin co-product from the transesterification section provided by line 248. In some instances, however, it may be desired to add additional base catalyst to third pretreatment reactor 148 or any preceding base pretreatment reactor. The pressure is typically in the range of between about 90 to 1000 kPa (absolute) although higher and lower pressures can be used. The reactor is typically batch, semi-batch, plug flow or continuous flow tank with some agitation or mixing. The preferred types of vessels are mechanical and sonically agitated reactors, and reactors with static mixing such as reactors containing contact structures such as trays, packing, baffles, orifices, venturi nozzles, tortuous flow path, and other impingement structures. Suitable reactors include those providing high intensity mixing, including high shear. However, depending upon the presence of soaps and phospholipids, care needs to be taken so as not to generate a product that cannot be readily separated by phase separation. The residence time will depend upon the desired degree of conversion, the ratio of alkanol to glyceride, reaction temperature, the degree of agitation and the like, and is often in the range of about 0.1 to 20, say, 0.5 to 10, hours.

Typically the transesterification in third pretreatment reactor 148 recovers through transesterification and extraction to the glyceride-containing phase at least about 20, preferably at least about 30, and more preferably at least about 50, mass percent of the alkanol fed to the reactor. Any unreacted alkanol in the oil phase will be carried with the oil phase to the transesterification component of facility 100. Often the total amount of alkanol recovered from the glycerin-coproduct from transesterification using all pretreatment stages is at least about 50, and sometimes at least about 80, mass percent. The third pretreatment product passes from third pretreatment reactor 148 through line 150 to separator 152. Separator 152 may be of any suitable design including a decanter, a phase separation facilitated decanter that contains coalescing sites, and, optionally, a centrifuge. Separator 152 serves to separate an oil phase containing glycerides, esters and alkanol and some catalyst, from a glycerin-containing phase containing glycerin, reduced concentration of alkanol, and catalyst. The glycerin-containing phase frequently contains less than about 15 mass percent alkanol. The glycerin-containing phase from separator 152 is passed via line 154 to second pretreatment reactor 139.

Facility 100 includes a chiller 158 to remove high molecular weight glycerides, waxes and esters that are insoluble at the chiller temperature. Some feeds, such as crude corn oil, contain high molecular weight glycerides and esters. The hydrocarbyl moieties in these high molecular weight components typically have between 30 and 40 carbon atoms. If they remain in the oil, the resultant biodiesel product tends to have unacceptably high cloud points and gel points. As shown, the oil phase from separator 152 passes through line 156 to chiller 158. Chiller 158 is maintained at a temperature sufficient to cause high molecular weight and other components that lead to and increase in gel point temperature to solidify. Typically this temperature is between about 0° C. and 20° C. In some instances, cooling will tend to remove monoglycerides and diglycerides. Cooling below the desired temperature and then warming to a temperature to liquefy the mono- and di-glycerides while still maintaining a solid wax, can minimize loss of components that can be converted to biodiesel. The chilled oil phase is then passed via line 160 to centrifuge 162 to remove higher density components including solids and any remaining glycerin phase. The higher density fraction is discharged via line 164. Rather than using a centrifuge, the solids can be filtered from the glyceride-containing stream. Filter aids can be used if desired. A producer composition is provided by centrifuge 162 and is provided to line 166.

Chiller 158 is optional, and chillers may also be used elsewhere in facility 100 to remove waxes. For instance, a chiller may be used to treat fresh feed in line 102 or can be used to treat biodiesel product from the refining component.

If desired all or a portion of the producer composition in line 166 may be withdrawn via line 168 as an intermediate product for storage or sale as a feedstock for transesterification. Line 168 also provides the feed for the transesterification component of facility 100 by introducing the producer composition into line 200.

Transesterification

Line 200 provides glyceride-containing feed to first transesterification reactor 202. Line 200 can also supply additional glyceride-containing feed. Preferably the additional feed is relatively free of free fatty acids and phospholipids such as refined oils sourced from rape seed, soybean, cotton seed, safflower seed, castor bean, olive, coconut, palm, corn, canola, fats and oils from animals, including from rendering plants and fish oils.

Alkanol for the transesterification is supplied to first transesterification reactor via line 206. The alkanol is preferably lower alkanol, preferably methanol, ethanol or isopropanol with methanol being the most preferred. The alkanol may be the same or different from the alkanol provided to esterification reactor 106 via line 124. Although line 206 is depicted as introducing alkanol into line 200, it is also contemplated that alkanol can be added directly to reactor 202 at one or more points. Generally the total alkanol (line 206 and from the producer composition of line 166) is in excess of that required to cause the sought degree of transesterification in reactor 202. Preferably, the amount of alkanol is from about 101 to 500, more preferably, from about 110 to 250, mass percent of that required for the sought degree of transesterification in reactor 202. In facility 100 three reactors are depicted as being used. One reactor may be used, but since the reaction is equilibrium limited, most often at least two and preferably three reactors are used. Often, where more than one reactor is used, at least about 60, preferably between about 70 and 96, percent of the glycerides in the feed are reacted in first transesterification reactor 202. It is possible to provide all the alkanol required for transesterification to first transesterification reactor 202, or a portion of the alkanol can be provided to each of the transesterification reactors.

The base catalyst is shown as being introduced via line 204 to first transesterification reactor 202. The amount of catalyst used is that which provides a desired reaction rate to achieve the sought degree of transesterification in first transesterification reactor 202. Preferably, catalyst is provided to each of the transesterification reactors since base catalyst preferentially partitions to the glycerin phase and is removed with phase separation of the glycerin after each transesterification reactor. The amount of catalyst used will be in excess of that required to react with the amount of free fatty acid contained in the feed oil, which due to the pretreatment, will be relatively little. The base catalyst may be an alkali or alkaline earth metal hydroxide or alkali or alkaline earth metal alkoxide, especially an alkoxide corresponding to the lower alkanol reactant. Preferred alkali metals are sodium and potassium. When the base is added as a hydroxide, it may react with the lower alkanol to form an alkoxide with the generation of water which in turn results in the formation of free fatty acid. Another type of catalyst is an alkali metal or alkaline earth metal glycerate. This catalyst converts to the corresponding alkoxide of the alkanol reactant in the reaction menstruum. Alternatively, the catalyst may be a heterogeneous base catalyst. Catalyst may need to be separately provided to the base pretreatment reactors if the base catalyst, e.g., a heterogeneous or oil soluble catalyst, is not carried with the co-product glycerin in the transesterification component to the base pretreatment reactors. However, homogeneous catalysts that have solubility in glycerin are preferred where the pretreatment component is used since the catalyst serves as at least a portion of the base used therein. The exact form of the catalyst is not critical to the understanding and practice of this invention. For the purposes of the following discussion, homogenous base catalyst is used. Preferably a non-acidic inerting gas such as nitrogen or hydrocarbon gas such as methane is used during base transesterification.

Often the transesterification is at a temperature between about 30° C. and 220° C., preferably between about 30° C. and 80° C. The pressure is preferably sufficient to maintain a liquid phase reaction menstruum and typically is in the range of between about 90 to 1000 kPa (absolute) although higher and lower pressures can be used. First transesterification reactor 202 is typically batch, semi-batch, plug flow or continuous flow tank with some agitation or mixing. Preferably the reactors are mechanical and sonically agitated reactors. Reactors with static mixing such as reactors containing contact structures such as trays, packing, baffles, orifices, venturi nozzles, tortuous flow path, and other impingement structures can be used. Suitable reactors include those providing high intensity mixing, including high shear. As stated above, one of the advantages of the processes of this invention is that the producer compositions do not require an induction period for the transesterification reaction to initiate. Accordingly plug flow reactors have enhanced viability. The residence time will depend upon the desired degree of conversion, the ratio of alkanol to glyceride, reaction temperature, the base catalyst concentration, the degree of agitation and the like, and is often in the range of about 0.02 to 20, say, 0.1 to 10, hours.

The partially transesterified effluent from reactor 202 is passed via line 208 to phase separator 210. Phase separator 210 may be of any suitable design including a decanter, a phase separation facilitated decanter that contains coalescing sites, and, optionally, a centrifuge. A glycerin-containing bottoms phase is provided in the separator and is removed via line 212 and is passed to glycerin header 214. As depicted, this stream is used as a portion of the glycerin for the pretreatment component of facility 100. This glycerin phase also contains any soaps made in reactor 202 and a portion of the catalyst. The soaps can be recovered from this stream in acidifying reactor 172 as discussed above. The lighter phase contains alkyl esters and unreacted glycerides and is passed via line 216 to second transesterification reactor 218. A rag layer may form in separator 210. The rag layer may contain unreacted glycerides, alkyl esters, alkanol, soaps, catalyst and glycerin. An advantage of the process set forth in FIG. 2 is that withdrawing the rag layer with the glycerin phase does not result in a loss of glycerides, alkyl esters, alkanol, and catalyst since the glycerin phase is passed to the pretreatment component of facility 100.

Reactor 218 may be of any suitable design and may be similar to or different than reactor 202. As shown, additional alkanol is provided via line 206A, and additional catalyst is provided via line 204A. Preferably the transesterification conditions in reactor 218 are sufficient to react at least about 90, more preferably at least about 95, and sometimes at least about 97 to 99.9 or more, mass percent of the glycerides in the feed to the transesterification. The transesterification in reactor 218 is typically operated under conditions within the parameters set forth for reactor 202 although the conditions may be the same or different. The residence time will depend upon the desired degree of conversion, the ratio of alkanol to glyceride, reaction temperature, the degree of agitation and the like, and is often in the range of about 0.02 to 20, say, 0.1 to 10, hours.

The effluent from second transesterification reactor 218 is passed via line 220 to phase separator 222 which may be of any suitable design and may be the same as or different from the design of separator 210. A heavier, glycerin-containing phase is withdrawn via line 224 and passed to glycerin header 214. A lighter phase containing crude biodiesel is withdrawn from separator 222 via line 226.

As depicted, third transesterification reactor 228 is used and the crude biodiesel in line 226 is passed to this reactor. The transesterification conditions in reactor 228 are sufficient to provide essentially complete conversion, at least about 97 or 98 to 99.9, mass percent of the glycerides in the feed converted to alkyl ester. As shown, additional alkanol is provided via line 206B, and additional catalyst is provided via line 204B. The transesterification in reactor 228 is typically operated under conditions within the parameters set forth for reactor 202 although the conditions may be the same or different. The residence time will depend upon the desired degree of conversion. The reactor may be of the type described for reactor 202. The residence time will depend upon the desired degree of conversion, the ratio of alkanol to glyceride, reaction temperature, the degree of agitation and the like, and is often in the range of about 0.02 to 20, say, 0.1 to 10, hours. Advantageously, the transesterification product from third transesterification reactor 228 contains less than about 1, preferably less than about 0.8, and most preferably less than 0.5, mass percent soaps based upon the total mass of alkyl esters and soaps. The lighter phase also contains alkanol. In reactor 228 the reaction proceeds quickly to completion by the addition of additional alkanol and catalyst, and can be conveniently accomplished by a plug flow reactor.

The overall molar ratio of alkanol to glycerides in the feed to the reactors in the transesterification component, i.e., alkanol provided by lines 206, 206A and 206B, can vary over a wide range. Since transesterification is an equilibrium-limited reaction, the driving force toward the alkyl ester and the conversion of glycerides will be dependent upon the molar ratio of alkanol equivalents to glycerides. Alkanol equivalents are alkanol and alkyl group of the alkyl esters in the feed to the transesterification component. On the basis of transesterfiable substituents in the feed to the transesterification component, the mole ratio of alkanol equivalents to glyceride in the feed to the pretreatment component is frequently between about 3.05:1 to 15:1, say 4:1 to 9:1. Advantageously, the pretreatment processes of this invention permit the reuse of alkanol partitioned to the co-product glycerin without intermediate vaporization. Often the amount of total catalyst provided based on the mass of feed to the first transesterification reactor, i.e., the catalyst provided by lines 204, 204A and 204B, is between about 0.3 and 1 mass percent (calculated on the mass of sodium methoxide).

The effluent from third transesterification reactor 228 is passed via line 230 to phase separator 232 which may be of any suitable design and may be the same as or different from the design of separator 210. A heavier, glycerin-containing phase is withdrawn via line 234 and passed to glycerin header 214. A lighter phase containing crude biodiesel is withdrawn from separator 232 via line 236. Alternatively, separator 232 can be eliminated provided that in second transesterification reactor 218, the conversion of the glycerides in the feed is at least about 90, preferably 92 to 96 or 98, percent. In some instances, the effluent from reactor 228 may be a single phase containing relatively little glycerin. In some instances it may be advantageous to use a centrifuge to separate the glycerin phase from the oil phase following third transesterification reactor 228.

Facility 100 contains an optional alkanol replacement reactor 238. The alkanol replacement reactor serves to transesterify the alkyl ester with a different alkanol. For purposes of transesterification in reactors 202, 218 and 228, an alkanol such as methanol provides not only attractive reaction rates but also an effluent that is more easily separated than, say, a reaction effluent where ethanol is the alkanol. In some instances it may be desired to provide a biodiesel that contains fatty esters in which the alkyl group of the fatty ester is branched in order to reduce cloud and gel points. The transesterification between, say, a fatty acid methyl ester, and higher molecular weight alkanol results in methanol, rather than glycerin, being formed, and often is more readily accomplished than the transesterification of glyceride with that higher alkanol. The higher alkanols include those having 2 to 8 or more carbon atoms, and are preferably branched primary and secondary alkanols although tertiary alkanols may find application but generally are less reactive. Examples of higher alkanols include propanol, isopropanol, isobutanol, 2,2-dimethylbutan-1-ol, 2,3-dimethylbutan-1-ol, 2-pentanol, and the like. Other alkanols include benzyl alcohol and 2 ethylhexanol.

Where an alkanol replacement operation is desired, it may be located at various points in the process. For instance, the replacement alkanol may be provided via line 206B to reactor 228, or, as shown, it can follow reactor 228. In either case, alkanol replacement transesterification can take advantage of catalyst contained in the transesterification medium. Alternatively, alkanol replacement may be effected on a biodiesel product by adding catalyst. Thus, it can be located elsewhere in the refining component of facility 100 including, but not limited to, treating biodiesel in line 352.

The amount of higher alkanol provided via line 240 to alkanol replacement reactor 238 can vary over a wide range. Typically the molar ratio of higher alkanol to alkyl ester being fed to reactor 238 is less than 0.5:1, e.g., from about 1:100 to 1:5. Often the alkanol replacement transesterification is at a temperature between about 30° C. and 220° C., preferably between about 30° C. and 80° C. The pressure is preferably sufficient to maintain a liquid phase reaction menstruum and typically is in the range of between about 90 to 1000 kPa (absolute) although higher and lower pressures can be used. Alkanol replacement reactor 238 can be batch, semi-batch, plug flow or continuous flow tank with some agitation or mixing, e.g., mechanically stirred, ultrasonic, static mixer containing contact surfaces, e.g., trays, packing, baffles, orifices, venturi nozzles, tortuous flow path, or other impingement structures. High intensity mixing reactors, including high shear reactors, may also be used. Preferred reactors are those in which the alkanol being replaced is continuously removed. For instance, a reactive distillation reactor can be used to continuously remove displaced methanol from a transesterification of methyl ester and isopropanol. As depicted, reactor 238 is a reactive distillation unit and lower alkanol is withdrawn via line 330A and passed to the transesterification reactors. Make-up alkanol is provided via line 332.

Where the alkanol replacement reactor is a batch reactor, driving the replacement reaction to either essentially complete conversion of the higher alkanol or essentially complete conversion of the methyl ester to the higher alkanol ester (depending upon whether the higher alkanol is provided below or at or above the stoichiometric amount required for complete conversion), since the vapor fractionation of methanol can continue until completion. With continuous reactors, having unreacted methanol and higher alkanol in the alkanol replacement product is likely. For purposes of this discussion, a continuous alkanol replacement reactor is used.

Where the base catalyst has been removed from the fatty acid ester of the lower alkanol, for instance, if the alkanol replacement were to be conducted on a refined or partially refined biodiesel, catalyst is provided. Suitable catalyst includes base catalyst such as is used for transesterification. Since a single liquid phase exists during the alkanol replacement unlike transesterification where a glycerin layer forms, heterogeneous catalysts and homogeneous catalysts having limited solubility in the reaction menstruum can be used. Solid catalysts are preferred to minimize or eliminate post treatment of the alkanol replacement product, but good contact with catalyst is desirable to timely achieve sought conversion. Homogeneous transesterification catalysts such as titanium tetra-isopropoxide are also advantageous as they are readily removed.

The residence time will depend upon the desired degree of conversion, the ratio of higher alkanol to alkyl ester, reaction temperature, the degree of agitation and the like, and is often in the range of about 0.02 to 20, say, 0.1 to 10, hours. Preferably at least about 80, and sometimes at least about 90, mass percent of the higher alkanol is reacted.

Refining

A crude biodiesel is withdrawn from reactor 238 via line 300 and is passed to the refining component of facility 100. The crude biodiesel may be contacted with acid to neutralize any catalyst therein and then refined to remove alkanol, soaps, water and glycerin.

In a preferred process, an acid, preferably an organic acid, is provided via line 302 in an amount sufficient to substantially neutralize residual base catalyst contained in the crude biodiesel. Inorganic acids such as sulfuric acid can be used as well as organic acids, particularly those less volatile than the alkanol, and acids that do not themselves or any potential reaction product formed in contact with the crude biodiesel, form azeotropes with the alkanol. Exemplary organic acids include acetic acid, citric acid, oxalic acid, glycolic, lactic, free fatty acid and the like. Generally the amount of catalyst contained in the crude biodiesel is quite small as base catalyst preferentially partitions to the glycerin phase. Accordingly, little acid is required to neutralize sufficient catalyst to enable refining without risk of reversion of alkyl ester. Often the amount of acid used is at least 0.95 times, sometimes between about 1 and 3 times, that required to neutralize the catalyst.

Crude biodiesel is passed via line 300 to an alkanol separation unit operation. As shown, a two stage separation unit is used. A single stage separator can be used if desired. The crude biodiesel in line 300 is passed to first alkanol separator stage 304. Separator 304 is of any convenient design including a stripper, wiped film evaporator, falling film evaporator, solid sorbent, and the like. Preferably the fractionation is by fast, vapor fractionation. Generally for a fast, vapor separation the residence time is less than about one minute, preferably less than about 30 seconds, and sometimes as little as 5 to 25 seconds. Preferably the vapor fractionation conditions comprise a maximum temperature of less than about 200° C., preferably less than about 150° C., and most preferably, when the lower alkanol is methanol, less than about 120° C. Depending upon the alkanol, the lower boiling fractionation may need to be conducted under subatmospheric pressure to maintain desired overhead and maximum temperatures. Where a falling film stripper is used, it may be a concurrent or countercurrent flow stripper. Concurrent strippers are preferred should there be a risk of undue vaporization of alkanol at the point of entry of the crude biodiesel. An inert gas such as nitrogen may be used to assist in removing the alkanol.

The fast fractionation may be effected by any suitable vapor fractionation technique including, but not limited to, distillation, stripping, wiped film evaporation, and falling film evaporation. Often the falling film evaporator has a tube length of at least about 1 meter, say, between about 1.5 and 5 meters, and an average tube diameter of between about 2 and 10 centimeters. Usually the vapor fractionation recovers at least about 70, preferably at least about 90, mass percent of the alkanol contained in the crude biodiesel. Any residual alkanol is substantially removed in any subsequent water washing of the crude biodiesel. Advantageously, the amount of alkanol contained in the spent water from the washing may be at a sufficiently low concentration that the water can be disposed without further treatment. However, from a process efficiency standpoint, alkanol can be recovered from the spent wash water for recycle to the transesterification reactors.

The lower boiling fraction containing the alkanol will contain a portion of any water contained in the crude biodiesel. Since the transesterification is conducted with little water being present, and a portion of the water is removed with the glycerin, the concentration of water in this fraction can be sufficiently low that it can be recycled to the transesterification reactors. This lower boiling fraction often contains less than about 1, and more preferably less than about 0.5, mass percent water. Alternatively, the lower boiling fraction may be passed to a methanol and water distillation column in the esterification section of facility 100.

Alkanol is exhausted from first alkanol separator stage via line 306 and may be exhausted from the facility as a by-product, e.g., for burning or other suitable use, or can be recycled. Where no alkanol replacement reaction is used, the alkanol will be the lower alkanol for the transesterification and is recycled to the transesterification section. The bottoms stream from first alkanol separation stage 304 is passed via line 308 to second alkanol separation stage 314 for additional alkanol recovery. The design of second alkanol separation stage 314 may be similar to or different than that of first alkanol separation stage 304 and may be operated under the same or different conditions. Alkanol exits via line 316 and is combined with alkanol from line 306 and is passed to condenser 318. In the process of facility 100, the condensed alkanol will contain both the lower alkanol and the higher alkanol. Condensed alkanol is recycled via line 330 to alkanol replacement reactor 238. Non-condensed gases exit condenser 318 via line 320. As shown, the alkanol separation operation is maintained under vacuum conditions and these gases are passed to liquid ring vacuum pump 322. The liquid for the liquid ring is provided via line 324 and exits via line 328. As the gases contain some alkanol, the liquid for the liquid ring vacuum pump will remove alkanol from the gases. The liquid may be water, in which case the water may need to be treated to remove alkanol. Alternative liquid streams can be used, including but not limited to glyceride-containing feed, biodiesel, and glycerin. Feed is preferred as the liquid for the liquid ring vacuum pump since it can be passed to a transesterification reactor and alkanol contained therein used for the transesterification. Gas is removed from liquid ring vacuum pump 322 via line 326.

The bottoms stream from the second alkanol separation stage exits via line 334 and is passed to separator 336 in which a glycerin-containing phase and a biodiesel-containing phase are separated. The presence of alkanol in the crude biodiesel enhances the solubility of glycerin therein. Upon removal of the alkanol, a separate glycerin-containing phase, which also contains soaps, tends to form during the alkanol separation operation. The glycerin fraction is removed from separator 336 via line 338 and can be combined with spent glycerin in line 186. The lighter, oil-containing phase is passed via line 340 to a water wash unit operation. If desired, techniques can be used to assist in the phase separation of glycerin in separator 336 such as adding an effective amount of water to assist in the separation. Other components useful in enhancing phase separations may also be used including water-soluble inorganic salts that are essentially insoluble in the biodiesel-containing phase. If desired, any water-containing phase can be passed to evaporator 374.

Line 340 serves as a reactor and mixer where strong acid is supplied. The amount of strong acid provided is sufficient to convert any soaps remaining to free fatty acids. Sufficient strong acid is used such that water used for washing the crude biodiesel is at a suitably low pH. The strong acid is supplied in admixture with a recycle stream in the wash operation as will be explained later. While line 340 serves as an in-line mixer, a separate vessel may be used for the acidification. Where a separate mixer is used, it may be of any convenient design, e.g., a mechanically or sonically agitated vessel, or static mixer containing static mixing devices such as trays, packing, baffles, orifices, venturi nozzles, tortuous flow path, or other impingement structure. In any event, sufficient mixing and residence time should be provided such that essentially all of the soaps are converted to free fatty acids. Often the temperature during the mixing is in the range of about 30° C. to 220° C., preferably between about 60° C. to 180° C., and for a residence time of between about 0.01 to 4, preferably 0.02 and 1, hours.

For purposes of discussion only and not in limitation, the water wash operation uses a two stage water wash. Water wash operation may be of any suitable design. Typically, the water wash operates with a recycling water loop, often with the water recycle being at least about 20, say between about 30 and 500, mass percent of the crude biodiesel being fed to the column. Normally washing is operated at a temperature between about 20° C. and 120° C., preferably between about 35° C. and 90° C. The amount of water provided to each wash vessel is sufficient to effect a sought removal of glycerin, residual alkanol and any water-soluble contaminants from the crude biodiesel. Typically between about 20 and 200, preferably between about 30 and 100, mass parts of wash water are used per 100 mass parts of crude biodiesel. Usually the free fatty acid is present in an amount less than about 3000, most frequently less than about 2500, parts per million by mass in the biodiesel product, and thus no need exists to remove free fatty acid to provide a biodiesel product meeting current commercial specifications. Preferably between about 1000 and 2500 ppm-m free fatty acid is contained in the biodiesel product to aid in lubricity.

The vessels used for the water washing may be of any suitable design including a pipe reactor, mechanically or sonically agitated tank, a vessel containing static mixing devices such as trays, packing, baffles, orifices, venturi nozzles, tortuous flow path, or other impingement structure. Each stage needs to effect a phase separation of the oil phase from the water phase. Such a separation may be inherent in, for instance, a wash column where the water and oil phases are moving countercurrently, or a separate phase separator may be provided. It is understood that other washing operations can be used such as a one vessel washing operation, an acid wash followed by a neutral wash, and the like. The washing may be effected in one or more stages and in one or more vessels. A single vessel, such as a wash column can contain a plurality of stages.

As shown, crude biodiesel is provided via line 340 to first wash stage 342. For purposes of discussion, wash stage 342 comprises an agitated vessel to provide desired contact between the oil and water phases and a decanter to effect separation. Typically, the agitated vessel provides a contact time of about 1 second and 10 minutes, say, 5 to 60 seconds. Crude biodiesel is contacted with acidic water from water loop 368. The washed biodiesel from first wash stage 342 is passed via line 344 to second wash stage 346 having a design similar to or different from that of stage 342. This biodiesel is contacted with water from water loop 364. In each stage the water, after contacting the biodiesel stream being processed, is returned to the respective loops. Acidic water is withdrawn from first wash stage 342 and recycled via line 368. Substantially neutral water is withdrawn from second wash stage 346 and recycled via line 364. Additional water is provided to line 364 via line 376 which will be described later.

As configured with separate water cycle loops, the pH of the water in second wash stage 346 may be neutral or less acidic than the water in first wash stage 342. Make-up water to line 368 is provided by line 366. A purge is taken from line 368 via line 372. The purge balances the amount of water in the wash loops and is at a suitable rate to maintain desirably low concentrations of impurities such as alkanol and glycerin in the water used for the washing. The purge is usually at a rate of between about 1 and 50, say 5 and 20, mass percent per unit time of the recycle rate in the loop.

Line 370 provides strong acid to the water recycled via line 368 for combining with crude biodiesel in line 340 or being passed to first wash stage 342. Adequate strong acid aqueous solution is provided that the water in line 368 has a pH sufficiently low to convert the soaps to free fatty acids. The acid may be any suitable acid to achieve the sought pH such as hydrochloric acid, sulfuric acid, sulfonic acid, phosphoric acid, perchloric acid and nitric acid. Sulfuric acid is preferred due to cost and availability and it is a non-oxidizing acid. The amount of strong acid aqueous solution provided is typically in a substantial excess of that required to convert the soaps to free fatty acid and to neutralize any remaining catalyst. The excess of acid is often at least about 5, preferably at least about 10, say between about 10 and 1000 times that required. Consequently the feed to first wash stage 342 provides a wash water in line 368 having a pH of up to about 4, preferably between about 0.1 and 4.

Returning to line 372, the purge water is passed to evaporator 374 which provides a lower boiling fraction and a higher boiling fraction. While an evaporator may be used, it is also possible to use a packed or trayed distillation column with or without reflux. Generally the bottoms temperature of evaporator 374 is less than about 150° C., preferably between about 120° C. and 150° C. The distillation may be at any suitable pressure. A membrane separation system may, alternatively or in combination, be used with evaporator 374 to effect the sought concentration of the spent water.

The lower boiling fraction contains water, potentially acid if not neutralized or salts, and some alkanol and is passed via line 376 to water wash loop 364. Fresh water is provided to line 376 by line 380. The higher boiling fraction contains glycerin, some alkanol and some water and potentially acid or salts thereof. The higher boiling fraction or a portion thereof is preferably passed via line 382 to line 170 or it can be combined with spent glycerin.

A washed biodiesel stream is withdrawn from second washing stage 346 via line 348 and is passed to drier 350 to remove water which exhausts via line 354. Preferably substantially all the alkanol has been removed from the crude biodiesel prior to drying to permit the water vapor to be exhausted without treatment to eliminate volatile organic components. Drier 350 may be of any suitable design such as stripper, wiped film evaporator, falling film evaporator, and solid sorbent. Generally the temperature of drying is between about 60° C. and 220° C., say, about 70° C. and 180° C. The pressure is generally in the range of about 5 to 200 kPa absolute. The dried biodiesel is withdrawn as product via line 352. The biodiesel product contains free fatty acid and preferably has a free fatty acid content of less than about 0.3 mass percent. An inert gas such as nitrogen may be used in facilitating drying.

The subatmospheric pressure is maintained in drier 350 by the use of liquid ring vacuum pump 356 which is in communication with line 354. Liquid ring vacuum pump 356 uses water as the sealing fluid which is provided by line 358 and water exits via line 362. The gases from liquid ring vacuum pump 356 exit via line 360.

Returning to glycerin header 214, the glycerin-containing streams are passed via line 242 to blending tank 246 such that a relatively uniform glycerin composition can be provided via line 248 to the pretreatment section of facility 100. Blending tank 246 may also provide sufficient residence time for any glycerides in the glycerin to transesterify with alkanol as well as permit any oil entrained in the glycerin phase to separate. As shown, an oil layer that forms in blending tank 246 can intermittently or continuously be withdrawn via line 247 for recycle to first transesterification reactor 202. Alternatively, the oil layer can be withdrawn with the glycerin and passed to the pretreatment section.

While all glycerin-containing streams from the transesterification and refining components of facility 100 have been shown to be directed to glycerin header 214, it is within the purview of the process to use fewer streams. As stated above, the bottoms from evaporator 374 may be passed via line 382 to line 170 or added to header 214 or removed from the facility as a by-product. Moreover, any of the glycerin-containing streams may be used elsewhere prior to being passed to blending tank 246, and the blended stream or a portion thereof in line 248 may be used elsewhere and either returned to glycerin header 214 or passed to pretreatment component of facility 100.

As discussed in connection with FIG. 2, the solvent may be obtained at various points within a biodiesel production facility which can operate as an acid catalyzed esterification process as well as a base catalyzed esterification process. The point or points at which the solvent is obtained will depend, among other things, concentration of glycerides acceptable in the solvent. Where the fermentation broth contains glycerides, and the solvent is recycled between extraction and distillation, it is generally preferred that the total glyceride content of the fresh solvent be less than about 50, preferably less than about 40, mass percent to reduce the amount of purge of solvent needed to be taken to maintain steady state conditions. The presence of minor amounts of water and of glycerin in the solvent is not usually deleterious.

As discussed in connection with FIG. 2, the solvent may be obtained at various points within a biodiesel production facility which can operate as an acid catalyzed esterification process as well as a base catalyzed esterification process. The point or points at which the solvent is obtained will depend, among other things, concentration of glycerides acceptable in the solvent. Where the fermentation broth contains glycerides, and the solvent is recycled between extraction and distillation, it is generally preferred that the total glyceride content of the fresh solvent be less than about 50, preferably less than about 40, mass percent to reduce the amount of purge of solvent needed to be taken to maintain steady state conditions. The presence of minor amounts of water and of glycerin in the solvent is not usually deleterious.

### Discussion of Other Figures

For purposes of illustrating the breadth of the invention and not in limitation thereof, various extraction systems will be described in connection with the remaining drawings.

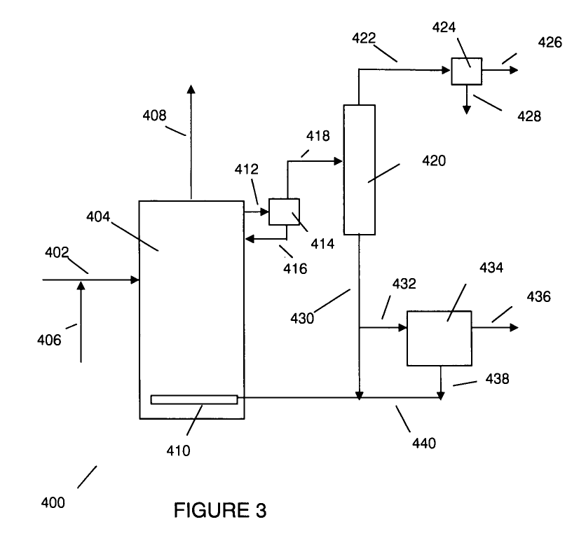

With respect to FIG. 3, an apparatus is depicted in which fermentation and solvent extraction of alkanol and glycerides simultaneously occur. As shown, carbohydrate source containing glycerides is provided via line 402 to fermentation vessel 404. Yeast and nutrients are provided to fermentation vessel 404 via line 406. Carbon dioxide is withdrawn from fermentation vessel 404 via line 408. Sparger 410 introduces solvent into vessel 404. Sparger 410 may be of any suitable design and is intended to distribute solvent across the cross-section of fermentation vessel 404. The sparger can be an annular ring. Alternatives include an annular ring on the interior surface of the vessel wall and a cross or star-burst shape distributor. The ports are contained both on the exterior and interior lateral sides of the annular ring. The sparger may also be a straight pipe with ports. Frequently the ports are sized such that the feed is introduced at a relatively low velocity, e.g., less than about 1 or 2, preferably less than about 0.7, say, between about 0.1 and 0.7, meter per second. The number of ports and placement are such that good distribution across the cross section of vessel 404 is obtained. Typically the ports have a diameter of less than about 2, say, about 0.5 to 1.5, centimeters. If the solvent is used to create mixing in the vessel, the ports may be of smaller size and directed such that the pressure of the entering solvent affects the sought mixing patterns.

The solvent rises through the fermentation broth in fermentation vessel 404 and collects at the top of the vessel. A stream containing solvent laden with alkanol and containing entrained fermentation broth is withdrawn from the top of vessel 404 and is passed via line 412 to phase separator 414. Phase separator 414 may be the same or different from that described as phase separator 128 in connection with FIG. 1. If desired, a centrifuge may be used to assure that no solids are contained in the solvent. The aqueous phase is returned to fermentation vessel 404 via line 416 and the solvent phase is passed via line 418 to flash distillation column 420.

The overhead from flash distillation column 420 is passed via line 422 to molecular sieve dryer 424. Product alkanol is provided via line 426 and water is removed via line 428. The bottoms from flash distillation column 420 are removed via line 430. A purge is taken from line 430 via line 432 and passed to biodiesel production unit 434. Biodiesel product exits biodiesel production unit 434 via line 436. A less refined, biodiesel-containing stream is withdrawn from biodiesel production unit 434 via line 438 and passed to line 440 which directs the solvent, including the recycle solvent from line 430, to sparger 410.

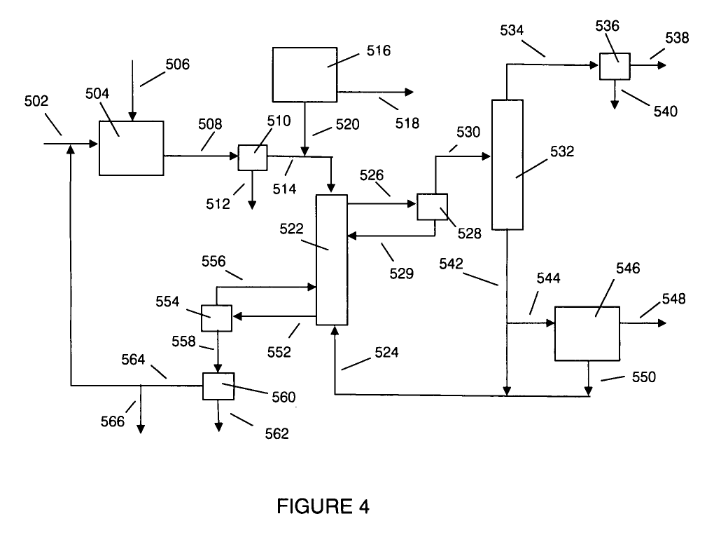

FIG. 4 illustrates a fermentation process in which a portion of the fermentation broth is removed from the fermentor and subjected to extraction with a solvent comprising biodiesel. For purposes of ease of understanding, the apparatus will be discussed in terms of the alkanol being ethanol although other alkanols could be made using the apparatus.

Carbohydrate source is provided via line 502 to fermentation vessel 504. The carbohydrate may be pretreated such as by wet milling or dry milling and enzymatic hydrolysis. For sake of illustration only, the source will be dry milled corn. Yeast and nutrients are provided to fermentation vessel via line 506. Fermentation vessel 504 may be an agitated vessel. Carbon dioxide is produced during the fermentation and is withdrawn from fermentation vessel 504. Fermentation broth is withdrawn via line 508.

The withdrawn fermentation broth contains ethanol, water, solids (distillers grains) and crude corn oil. As shown, it is passed to centrifuge 510 where wet solids are removed via line 512. The liquid phase is then passed via line 514 to extraction column 522 where it is contacted with solvent, which is biodiesel, e.g., a methyl ester of corn oil, provided by line 524. Extraction column 522 may be of any suitable design. As shown, extraction column 522 is adapted to provide for countercurrent contact between the solvent and aqueous phases. The column may be a packed column to assist in liquid-liquid contact or may comprise a series of discrete stages, each with agitation to promote liquid-liquid contact, with phase separation therebetween. The extraction column serves to remove ethanol and glycerides contained in the corn oil.

Usually the fermentation broth contains between about 5 and 15, say, 6 and 12, mass percent ethanol. In one of the preferred embodiments, at least a portion of the aqueous phase remaining after extraction of the ethanol with water-immiscible solvent is recycled including with unconsumed carbohydrates and retained ethanol to the fermentation unit operation. Accordingly, for economically feasible production of the ethanol, it is not necessary to have essentially complete conversion of the carbohydrate per pass. Thus, more severe fermentation conditions that are required for essentially complete conversion can be avoided which can result in less generation of fermentation inhibitors. In this mode of operation, the net make of ethanol may be in the range of between about 30 to 95, say, 50 to 90, mass percent of the total ethanol in the fermentation broth. At the end of the run, the rate of introduction of solvent may be increased to reduce the concentration of ethanol in the fermentation broth. Alternatively, at the end of the run, the alkanol may be distilled from the broth. In one embodiment, the residual ethanol remains in the fermentation broth and the broth is treated to recover distillers grains and syrup. An ethanol and water mixture is thus obtained from the evaporators and driers and can be used in the pretreatment of the carbohydrate-containing feedstock and in the fermentation. The ethanol so recycled would be recovered through contact with the solvent.

The fermentation broth also contains solids including insoluble carbohydrate components, microorganism and other solid remains. Depending upon the feedstock to the fermentation unit operation, the fermentation broth can contain indigestible solids and liquids such as glycerides, cellulosics, and the like. Additionally fermentation inhibitors can be generated during the fermentation. Fermentation inhibitors are components that inactivate or kill the microorganism used for the fermentation. These components include, but are not limited to, the carboxylic acids corresponding to the ethanol being produced such as acetic acid, propionic acid, butyric acid, and the like.

Alternatively, at least a portion of the fermentation broth can be withdrawn from the fermentor for contact with the solvent. For instance in a batch fermentation, the entire fermentation broth at the completion of the fermentation operation can be contacted with the solvent. In a batch, semi-continuous and continuous fermentation, a portion of the fermentation broth can continuously or intermittently withdrawn for contact with the solvent. Where a portion of the broth is withdrawn for the contacting, normally the portion at any given time is from about 5 to 20 mass percent of the total fermentation broth. If desired, the aqueous fermentation broth after contact with the solvent, can be recycled to the fermentor.

Spent solvent is withdrawn from extraction column 522 via line 526 and passed to phase separator 528. Phase separator 528 may be of any suitable design including a centrifuge or a gravity vessel. The heavier aqueous phase is shown as being returned to extraction column 522 via line 529. The solvent phase is passed via line 530 to flash distillation column 532. An ethanol overhead is taken via line 534 from distillation column 532. Some water will be present in the ethanol. If desired, the overhead may be further treated to remove water in unit operation 536. Unit operation 536 may be, for instance, a molecular sieve dryer. Alternatively, unit operation 536 may be a distillation column if the amount of water present in the overhead is less than that which forms an azeotrope. Ethanol product is provided via line 538 from unit operation 536 and removed water is recovered via line 540. The water may be recycled to fermentation vessel 504 or to a unit operation pretreating the corn.

The higher boiling fraction from distillation column 532, distillation column bottoms fraction, contains biodiesel and extracted glycerides and is withdrawn via line 542. All or a portion of the higher boiling fraction is withdrawn from line 542 and passed to biodiesel production unit 546 for conversion to biodiesel. That portion not withdrawn can be passed to line 524 for recycle to extraction column 522. The glycerides can serve as solvent to remove ethanol and hence the amount withdrawn via line 544 need be only that to maintain a steady state operation with a desired concentration of glycerides in the feed to the biodiesel production unit. One type of biodiesel production unit employs a transesterification to convert glycerides to biodiesel and this transesterification is an equilibrium-limited reaction. Accordingly, it is generally desired that the portion of the higher boiling fraction from distillation column 532 that is withdrawn via line 544 be sufficient that the total glycerides comprise at least about 20, preferably between about 30 and 70, mass percent of the higher boiling fraction withdrawn from distillation column 532.

Biodiesel production unit 546 may be of any suitable type including acid catalyzed esterification and base catalyzed transesterification. A particularly attractive unit is that disclosed in copending International application No. PCT/US2007/020248, International filing date 19 Sep. 2007, published as WO 2008/036287, herein incorporated by reference. Any suitable lower alkanol may be used for the conversion of the glycerides. Methanol is typically chosen due to availability, cost and ease of separations within the biodiesel production unit. Due to the availability of ethanol, ethanol may be a preferred alkanol for the conversion of glycerides to the esters that comprise biodiesel. Either ethanol can be used as the alkanol or, as disclosed in U.S. Provisional Patent Application 60/994,454, filed 19 Sep. 2007, and International application No. PCT/US08/076,630, filed Sep. 17, 2008, herein incorporated in its entirety by reference, a methyl ester may be made and then exchanged with ethanol to provide an ethyl ester biodiesel with the liberated methanol being recycled.

As shown, biodiesel product is provided by line 548 from biodiesel production unit 546. This product is refined to meet biodiesel standards such as ASTM D6751, American Society for Testing and Materials. While a portion of this product can be used as the solvent, it is often more cost effective to use a less refined biodiesel intermediate from biodiesel production unit 546 such as prior to washing and drying. As shown, an intermediate biodiesel product is withdrawn from biodiesel production unit 546 via line 550 and passed to line 524 for use as solvent in extraction column 522.

Returning now to extraction column 522, an aqueous phase from which ethanol has been removed is withdrawn via line 552 and passed to phase separator 554. Phase separator 554 may be of any suitable design including a centrifuge or a gravity vessel. The lighter solvent phase is recycled via line 556 to extraction column 522. The heavier, aqueous phase is passed via line 558 to optional centrifuge 560 for removal of any solids. If it is desired to remove carboxylic acids by precipitation as salts, such can be done by introducing a suitable base such as calcium oxide or calcium hydroxide into line 558. Wet solids are removed from centrifuge 560 via line 562. The liquid from centrifuge 560 is passed via line 564 to fermentation vessel 504.

The wet solids from line 512 and from line 562 can be treated to remove ethanol and dried to provide distillers grains as a by-product from the ethanol plant facility. For instance, the wet solids can be provided to dryer 516 with the water and ethanol being withdrawn via line 520 and returned to line 514 for ethanol recovery in extraction column 522. Alternatively, the ethanol and water can be recycled to the fermentation vessel 504 or upstream unit operations for treating the corn. The dried solids are removed via line 518.

As shown, a purge is taken via line 566 from line 564. This purge can be processed in the same or different equipment from that used to dry the wet solids. For instance, the purge can be contacted with biodiesel to remove additional quantities of ethanol and then distilled to remove water from, e.g., unreacted carbohydrates and the like.

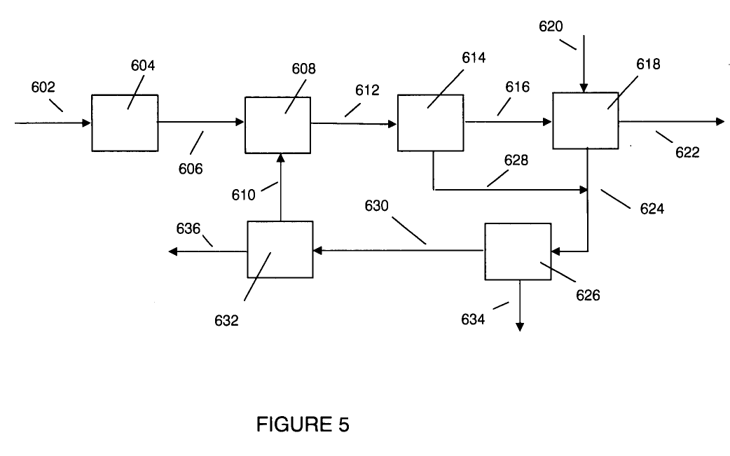

FIG. 5 depicts a process for extracting glycerides from soybean. Soybeans are provided via line 602 to flaker 604. Flaked soybeans are then passed via line 606 to agitated tank 608 where they are contacted with biodiesel-containing extractant provided by line 610. A slurry of extractant and solids from agitated tank 608 is passed via line 612 to crusher 614 where liquid is removed via line 628 and wet solids are passed via line 616 to centrifuge 618. Water is provided to centrifuge 618 via line 620 at a rate sufficient to displace extractant from the solids which are removed via line 622. The liquid phase, which is water and extractant containing glycerides is combined with the liquid from line 628 and passed to decanter 626. The aqueous phase exits via line 634 and the biodiesel-containing oil phase is passed via line 630 to biodiesel production facility 632 which provides extractant via line 610 and biodiesel product via line 636.

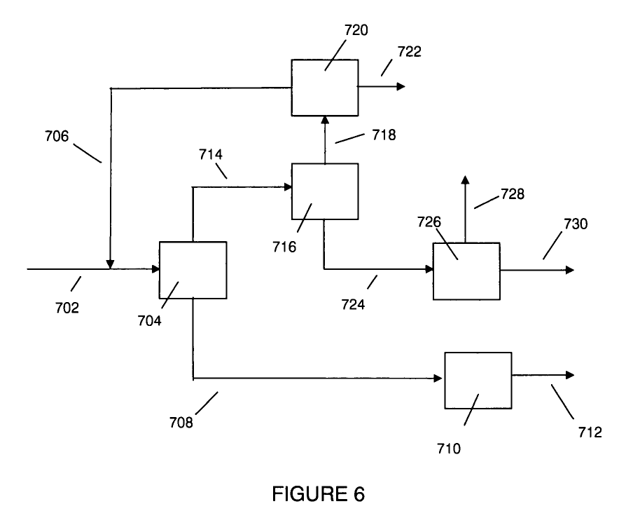

With respect to FIG. 6, whole stillage from an ethanol beer still is passed via line 702 to centrifuge 704. Prior to entering centrifuge 704, extractant containing biodiesel is added via line 706. Centrifuge 704 provides a wet distillers grains fraction which is passed via line 708 to dryer 710. Any residual extractant and glycerides contained in the wet distillers grains will remain with the distillers grains during the drying and be contained in the dried distillers grains product removed via line 712. As glycerides and biodiesel are non-toxic, the minor amount that remains with the distillers grains product poses no toxicity concerns for use of the product as an animal feed. Alternatively, the extractant can be added to the thin stillage produced by centrifuge 704 and recovered therefrom, thereby leaving a greater concentration of glycerides on the distillers grains. Due to the low viscosity of the extractant, the total fatty acid ester content (biodiesel and glycerides) of the distillers grains is generally less than that without the prior extraction of glycerides.

Centrifuge 704 provides a thin stillage that also contains extractant and glycerides. The thin stillage is passed via line 714 to centrifuge 716 that provides an extractant fraction withdrawn via line 718 and is sent to biodiesel facility 720 which provides biodiesel product via line 722 and extractant via line 706. Centrifuge 716 also provides an aqueous fraction containing carbohydrates which is passed via line 724 to evaporator 726 to remove water via line 728 and provide a syrup that exits via line 730. The water in line 728 can be recycled. The syrup can be added to the distillers grains to provide dry distillers grains with solubles or can be used as a fuel or intermediate product.

One advantage of this embodiment is that the feed to the evaporator contains relatively little glycerides and thus reduces energy required for the evaporation due to the reduction in the volume of the syrup. In the evaporation, the energy required for drying the syrup increases as the viscosity of the syrup increases. Additionally, the production of free fatty acids is reduced since glycerides are not subjected to higher temperatures and water which exist in the evaporator.

The addition of the extractant is shown as being to the whole stillage. By the use of extractant, glycerides can be removed without undue formation of emulsions with the thin stillage during centrifuging to remove glycerides.

It is within the broader aspects of the invention that the extractant can be added to the thin stillage with phase separation prior to evaporation, or to the syrup with phase separation, especially by centrifuging. The distillers grains can be treated with biodiesel to remove glycerides.

### EXAMPLES

The following example is by way of illustration of the invention and is not intended to be in limitation thereof.

A syrup is obtained from an evaporation stage removing water from thin stillage from an ethanol plant. The syrup is highly viscous and contains about 5.5 mass percent glycerides and free fatty acids. A first portion is centrifuged at about 70° C. for 20 minutes and an oil phase containing about 55 mass percent of the oil in the syrup is obtained. A second portion of the syrup is contacted with 10 parts by mass biodiesel and stirred to form a liquid dispersion. Due to the hydrophobic nature of the biodiesel, the dispersion is poorly formed. The liquid is centrifuged at about 70° C. for 20 minutes and an oil phase containing about 75 mass percent of the oil in the syrup is obtained.

It is claimed:

1. An integrated process recovery of glycerides from biomass derived feedstock, said feedstock containing glycerides and water, and making biodiesel comprising:
(a) contacting the biomass derived feedstock with a water-immiscible extractant comprising biodiesel under conditions such that at least a portion of the glycerides in the feedstock pass to the extractant to provide an extract containing biodiesel and glycerides and to provide a feedstock having a reduced concentration of glycerides;
(b) phase separating the extract from the feedstock;
(c) subjecting at least a portion of the extract in the presence of lower alkanol to ester forming conditions to convert glycerides to biodiesel and coproduce glycerin; and
(d) recycling a portion of the biodiesel to step (a) as at least a portion of the extractant.

2. The process of claim 1 wherein the biomass derived feedstock is from at least one of rape seed, soybean, cotton seed, safflower seed, castor bean, olive, coconut, palm, corn, canola, jatropha, rice bran, tobacco seed, fats and oils from animals.

3. The process of claim 2 wherein the biomass derived feedstock is milled, ground or flaked soybean.

4. The process of claim 2 wherein the biomass derived feedstock is from a process stream in a process for the production of ethanol by fermentation of carbohydrate.

5. The process of claim 4 wherein the process stream contains fermentation broth.

6. The process of claim 4 wherein the process stream contains distillers grains.

7. The process of claim 4 wherein the process stream is a thin stillage after separation of distillers grains.

8. The process of claim 4 wherein the process stream contains hydrolyzate of carbohydrate.

9. The process of claim 1 wherein the ester forming conditions comprise acid catalyzed esterification.

10. The process of claim 1 wherein the ester forming conditions comprise base catalyzed transesterification.

11. An integrated alkanol fermentation and biodiesel production process comprising:
(a) contacting at least a portion of a fermentation broth containing alkanol, glycerides and water with extractant comprising biodiesel, said contacting being for a time and under conditions including the mass ratio of extractant to broth to provide an extract containing glycerides and alkanol and to provide an aqueous phase having a reduced concentration of said alkanol and glycerides;
(b) phase separating the extract and the aqueous phase;
(c) separating by distillation alkanol from extract to provide an alkanol fraction and an extract fraction containing biodiesel and glycerides;
(d) subjecting at least a portion of the extract fraction in the presence of lower alkanol to ester forming conditions to convert glycerides to biodiesel and coproduce glycerin; and
(e) recycling a portion of the biodiesel to step (a) as at least a portion of the extractant.

12. The process of claim 11 wherein in step (a) extractant is introduced into the fermentation broth during fermentation.

13. The process of claim 12 wherein in step (a) extractant is contacted with at least a portion of the fermentation broth that has been removed from fermentation.

14. An integrated alkanol fermentation and biodiesel production process comprising:
(a) fermenting carbohydrate in an aqueous fermentation broth to produce alkanol, said fermentation broth containing glycerides and solids;
(b) subjecting at least a portion of the fermentation broth to distillation conditions to remove at least a portion of the alkanol and provide a whole stillage;
(c) contacting the whole stillage with extractant comprising biodiesel to provide an extract containing glycerides and an aqueous phase having a reduced concentration of glycerides and solids;
(d) phase separating the whole stillage to provide a solids-containing phase and at least one liquid phase;
(e) phase separating the extract and the aqueous phase;
(f) subjecting at least a portion of the extract in the presence of lower alkanol to ester forming conditions to convert glycerides to biodiesel and coproduce glycerin; and
(g) recycling a portion of the biodiesel to step (c) as at least a portion of the extractant.

15. The process of claim 14 wherein steps (d) and (e) are simultaneously performed.

16. An integrated alkanol fermentation and biodiesel production process comprising:
(a) fermenting carbohydrate in an aqueous fermentation broth to produce alkanol, said fermentation broth containing glycerides and solids;
(b) subjecting at least a portion of the fermentation broth to distillation conditions to remove at least a portion of the alkanol and provide a whole stillage;
(c) phase separating the whole stillage to provide a solids-containing phase and a thin stillage;
(d) contacting the thin stillage with extractant comprising biodiesel to provide an extract containing glycerides and an aqueous phase having a reduced concentration of glycerides;
(e) phase separating the extract and the aqueous phase;
(f) subjecting at least a portion of the extract in the presence of lower alkanol to ester forming conditions to convert glycerides to biodiesel and coproduce glycerin; and
(g) recycling a portion of the biodiesel to step (d) as at least a portion of the extractant.

17. The process of claim 16 wherein the thin stillage is subjected to evaporation conditions prior to step (d).

18. The process of claim 16 wherein the phase separation of step (e) comprises centrifugation and sufficient biodiesel is provided to avoid undue emulsion formation during separation.

19. An integrated process recovery of glycerides from biomass derived feedstock, said feedstock containing glycerides, water and solids, and making biodiesel comprising:
(a) contacting the biomass derived feedstock with a water-immiscible extractant comprising biodiesel under conditions such that at least a portion of the glycerides in the feedstock pass to the extractant to provide an extract;
(b) phase separating the extract from the solids wherein the solids contain residual extractant;
(c) contacting the solids with at least one of water and steam under conditions sufficient to remove at least a portion of the residual extract from the solids and provide a wash liquid contain water and extract;
(d) phase separating the wash liquid to provide an extract-containing fraction and an aqueous fraction;
(e) subjecting at least a portion of the separated extract from steps (b) and (d) in the presence of lower alkanol to ester forming conditions to convert glycerides to biodiesel and coproduce glycerin; and
(f) recycling a portion of the biodiesel to step (a) as at least a portion of the extractant.

20. The process of claim 19 wherein biodiesel is phase separated from glycerin prior to step (f).
